(19) United States
(12) Patent Application Publication
(10) Pub. No.: US 2026/0097056 A1
(43) Pub. Date: Apr. 9, 2026

(54) SYK ACTIVATORS AND INHIBITORS IN NEURODEGENERATIVE DISEASES

(71) Applicant: University of Virginia Patent Foundation, Charlottesville, VA (US)

(72) Inventors: John R. Lukens, Palo Alto, CA (US); Hannah Elzabeth Ennerfelt, Palo Alto, CA (US); Elizabeth Frost, Ruckersville, VA (US)

(21) Appl. No.: 19/112,913

(22) PCT Filed: Sep. 20, 2023
(86) PCT No.: PCT/US2023/074652

§ 371 Date: Mar. 18, 2025

Related U.S. Application Data
(60) Provisional application No. 63/376,425, filed on Sep. 20, 2022.

(51) Int. Cl. A61K 31/702; A61K 31/05; A61K 31/36; A61K 31/437; A61K 31/4985; A61K 31/501; A61K 31/506; A61K 31/519; A61K 31/5377; A61K 31/5383; A61K 31/675; A61P 25/28

(52) U.S. Cl. CPC A61K 31/702; A61K 31/05; A61K 31/36; A61K 31/437; A61K 31/4985; A61K 31/501; A61K 31/506; A61K 31/519; A61K 31/5377; A61K 31/5383; A61K 31/675; A61P 25/28

(57) ABSTRACT

Provided herein are methods to treat neurodegenerative disease.

### CROSS-REFERENCE TO RELATED APPLICATIONS

This application claims the benefit of U.S. Provisional Application No. 63/376,425, filed Sep. 20, 2022, the content of which is herein incorporated by reference in its entirety.

### GOVERNMENT SUPPORT

This invention was made with Government support under Grant No. RF1AG071996 awarded by the National Institute of Health (NIH). The Government has certain rights in this invention.

### INCORPORATION BY REFERENCE OF SEQUENCE LISTING

This application contains a sequence listing. It has been submitted electronically as an XML file titled “1036331WO1.xml.” The sequence listing is 2,621 bytes in size and was created on Sep. 19, 2023. It is hereby incorporated by reference in its entirety.

### BACKGROUND

Neurodegenerative disorders, such as Alzheimer's disease (AD), are major public health issues that are likely to increase in prevalence with the aging population. In general terms, neurodegenerative diseases are thought to be driven by the accumulation of neurotoxic material such as amyloid beta (Aβ) or myelin debris in the central nervous system (CNS) (Nussbaum and Ellis, 2003; Trapp and Nave, 2008). The buildup of neurotoxic agents is believed to cause neuronal damage and death, which can ultimately lead to various forms of neurological dysfunction that include cognitive decline, motor abnormalities, mental disorders, and loss of inhibition (Chung et al., 2018; Taylor et al., 2002; Vickers et al., 2009).

### SUMMARY OF THE INVENTION

Many neurodegenerative diseases are thought to be caused by impaired containment and/or disposal of neurotoxic material such as amyloid beta (Aβ) and myelin debris. Indeed, recent human genome-wide association studies (GWAS) and animal model studies have begun to reveal roles for the brain's professional phagocytes, microglia, as well as various innate immune receptors expressed by microglia in the control of neurotoxic material and subsequent neurodegenerative disease pathogenesis. Yet, the intracellular molecules that orchestrate the neuroprotective functions of microglia in degenerative disorders remain poorly understood. Provided herein is the identification of the innate immune signaling molecule spleen tyrosine kinase (SYK) as a regulator of microglial phagocytosis in neurodegenerative disease. It was found that targeted deletion of SYK in microglia leads to exacerbated Aβ deposition, aggravated neuropathology, and cognitive defects in the 5xFAD mouse model of Alzheimer's disease (AD). Furthermore, disruption of SYK signaling in this AD model was also shown to impede the development of disease-associated microglia (DAMs), alter AKT/GSK3β-signaling in microglia, and to cause severe deficits in the ability of microglia to phagocytose Aβ. These neuroprotective functions of SYK in microglia were not only restricted to Aβ-driven models of neurodegeneration, as it was found that SYK is also a regulator of microglial phagocytosis and DAM phenotype acquisition in demyelinating disease. Collectively, these results help to break new ground in the understanding of the innate immune signaling molecules that instruct beneficial microglial functions in response to neurotoxic material. Moreover, these findings suggest that targeting SYK may offer a therapeutic strategy to treat a spectrum of neurodegenerative disorders. It was further found that inhibiting SYK was beneficial in tauopathies.

One embodiment provides a method to treat or prevent Aβ-driven neurodegeneration or a demyelinating disease comprising administering an activator of SYK to a subject in need thereof. Another aspect provides method to enhance protective microglial activities (such as phagocytosis of neurotoxic material) comprising administering an activator of SYK to a subject in need thereof. One aspect provides a method to activate microglial activities/cells comprising administering an activator of SYK to a subject in need thereof. One aspect provides a method to increase clearance of myelin debris comprising administering an activator of SYK to a subject in need thereof. Another aspect provides a method to preserve neuronal health comprising administering an activator or inhibitor of SYK to a subject in need thereof. One aspect provides method to prevent a generate an increase in spatial learning and/or memory in Aβ-driven neurodegeneration or a demyelinating disease comprising administering an activator of SYK to a subject in need thereof. One aspect provides a method to prevent neuronal loss in Aβ-driven neurodegeneration or a demyelinating disease comprising administering an activator of SYK to a subject in need thereof. In one aspect, the subject is a mammal, such as a human. In one aspect the SYK activator is a β-D-glucan. In one aspect the SYK activator is a ligand for CLEC7A. In one aspect, the SYK activator is pustulan. In one aspect, the Aβ-driven neurodegeneration is Alzheimer's Disease (AD). In another aspect, the demyelinating disease is multiple sclerosis (MS) Balo's disease, acute-disseminated encephalomyelitis, progressive multifocal leukoencephalopathy, Charcot-Marie-Tooth Disease, Guillain-Barre Syndrome (GBS), HTLV-I Associated Myelopathy (HAM), Neuromyelitis Optica (Devic's Disease), Schilder's Disease, Transverse Myelitis or extrapontine myelinolysis.

One aspect provides a method to treat or prevent a tauopathy comprising administering an inhibitor of SYK to a subject in need thereof. In one aspect, the subject is a mammal, such as a human. In one aspect, the SYK inhibitor comprises GSK 143, Gusacitinib, R406, Fostamatinib, Piceatannol, Dehydroabietic acid, Cerdulatinib, MNS (3,4-Methylenedioxy-β-nitrostyrene), R112, BAY-61-3606, RO9021, Entospletinib, PRT-060318 2HCl, PRT062607 (P505-15) HCl, TAK-659, SRX3207, Lanraplenib or salts, compositions and combinations thereof. In aspect, the tauopathy comprises Pick disease, progressive supranuclear palsy, corticobasal degeneration, argyrophilic grain disease, frontotemporal lobar degeneration (FTLD-Tau), Alzheimer disease (AD; a secondary tauopathy), chronic traumatic encephalopathy, post-encephalitic parkinsonism or Parkinsonism linked to chromosome 17.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1I. Deletion of Syk in microglia leads to increased Aβ burden and altered plaque composition in 5xFAD mice (A-I) 5xFAD Syk^{fl/fl}Cx3cr1^{ERT2Cre} (5xFAD Syk^{ΔMG} mice) and Cre-negative 5xFAD Syk^{fl/fl} littermate controls (5xFAD mice) received tamoxifen food for 2 weeks starting at 3 weeks of age and then mice were returned to regular food for the remainder of the experiment. Mice were later harvested at 5 months of age to evaluate amyloid beta (Aβ) load in the brain. (A-B) Immunofluorescence staining of Aβ (D54D2, red; DAPI, blue) was performed on sagittal sections and the percent area covered by Aβ was quantified. (C) Sphericity of ThioflavinS (ThioS)-labeled and Imaris-rendered Aβ plaques in the cortex. (D) Quantification of sphericity with 1.00 being the most spherical, combined data from a total of 50-100 plaques from 3 matching brain sections per mouse. (E-F) Representative images and quantification of Aβ plaque composition labeling 6E10 (purple) for filamentous forms of Aβ and ThioS (blue) for more compact or inert forms of Aβ in the cortex. (F) Quantification represents the percent volume of the 6E10/ThioS ratio per field of view (FOV) from a total of 10-15 plaques from 3 brain sections per mouse. (G-I) Soluble (PBS) and insoluble (Triton-X and Guanidine) fractions of Aβ₁₋₄₀ and Aβ₁₋₄₂ measured by ELISA. Statistical significance between experimental groups was calculated by unpaired Student's t-test. *p<0.05, **p<0.01, ***p<0.001, ****p<0.0001 (B, D, F-I). Error bars represent mean±S.E.M. and each data point represents an individual mouse.

FIG. 2A-2I. Loss of Syk in microglia negatively affects neuronal health and exacerbates AD-related behaviors in 5xFAD mice (A-I) 5xFAD Syk^{ΔMG} mice and 5xFAD littermate controls received tamoxifen food for 2 weeks starting at 3 weeks of age and then mice were returned to regular food for the remainder of the experiment. (A-F) Brains were later harvested at 5 months of age to evaluate neuronal health and cell death. (A-B) The formation of dystrophic neurites surrounding plaques in the cortex was determined by staining for APP (blue) and Aβ using ThioS (pink). (B) Quantification of APP⁺ puncta found within 15 and 30 μm of Aβ plaques from a total of ˜40 plaques from 3 matching brain sections per mouse. (C-D) Cortical sections were stained with AT8 (yellow) for phosphorylated tau (p-tau) puncta found within 15 μm of ThioS (pink) stained Aβ plaques. (D) Quantification of p-tau from a total of ˜40 plaques from 3 matching sections per mouse. (E-F) Neuronal cell death in the CA1 region of the hippocampus was evaluated by TUNEL assay (green) and NeuN staining (pink). (F) Quantification of volume of TUNEL⁺ stain found in NeuN⁺ nuclei from 2 corresponding brain sections per mouse. (G-H) 4-month-old 5xFAD (n=6) and 5xFAD Syk^{ΔMG} (n=8) mice were evaluated in the Morris water maze (MWM). Statistics for MWM acquisition were calculated on day 4. Combined data from 3 independent experiments. (I) Performance in the elevated plus maze (EPM) was measured in 4-month-old 5xFAD and 5xFAD Syk^{ΔMG} mice. Combined data from 2 independent experiments. Statistical significance between experimental groups was calculated by unpaired Student's t-test (B, D, F-I). *p<0.05, **p<0.01, ***p<0.001. Error bars represent mean±S.E.M. and each data point represents an individual mouse.

FIG. 3A-3F. Syk-deficiency limits microglial proliferation and association with Aβ plaques (A-F) 5xFAD Syk^{ΔMG} mice and 5xFAD littermate controls received tamoxifen food for 2 weeks starting at 3 weeks of age and then mice were returned to regular food for the remainder of the experiment. Brains were later harvested at 5 months of age to evaluate microgliosis. (A-C) Microglia were imaged by labeling with Iba1 (green) surrounding Aβ plaques labelled with ThioS (pink) to assess microglial coverage and proximity to plaques. (A) Representative images of Iba1 and ThioS staining in the cortex. (B) Quantification of microglial numbers. (C) Quantification of microglial association with plaques as the percent of microglia within 15 μm of a plaque normalized to the total number of microglia. (D) Quantification of the number of microglia within a 15 and 30 μm radius surrounding ThioS-labelled Aβ plaques. Each point represents an individual mouse with an average of 50-100 plaques from 3 matching brain sections per mouse. (E) Representative images of microglial proliferation measured by evaluating Ki67 (blue) colocalization with Iba1+ (green) microglia in the cortex. (F) Quantification of Ki67+ microglia. Statistical significance between experimental groups was calculated by an unpaired Student's t-test. **p<0.01, ***p<0.001, ****p<0.0001. (B-D, F). Error bars represent mean±S.E.M. and each data point represents an individual mouse.

FIG. 4A-4H. Defective activation of Syk-deficient microglia in 5xFAD mice (A-H) 5xFAD Syk^{fl/fl}Cx3cr1^{ERT2Cre} (5×FAD Syk^{ΔMG} mice), Cre-negative 5xFAD Syk^{fl/fl} littermate controls (5xFAD mice), Syk^{fl/fl}Cx3cr1^{ERT2Cre} (Syk^{ΔMG} mice), and Cre-negative Syk^{fl/fl} littermate controls (Syk^{con} mice) received tamoxifen food for 2 weeks starting at 3 weeks of age and then mice were returned to regular food for the remainder of the experiment. Brains were later harvested at 5 months of age to evaluate microglial activation. (A) Imaris-rendered microglia morphology labeled with Iba1 (blue) in the cortex. (B) Sholl analysis quantification from a total of 12 microglia from 3 matching brain sections per mouse (5xFAD n=9, 5xFAD Syk^{ΔMG} n=8). (C-F) Microglia from 5-month-old mice were sorted from single-cell brain suspensions using anti-CD11b⁺-coated magnetic beads and magnetic column sorting, finally, RNA-Seq was performed. (C) Principal component (PC) analysis of sample clustering. (D) Volcano plots depicting differentially expressed genes (FDR<0.1). (E) KEGG term enrichment scatter plot highlighting major pathways that are repressed in 5xFAD Syk^{ΔMG} microglia in comparison to 5xFAD microglia. (F) Heatmap representation of significantly downregulated (FDR<0.1) stage 1 & 2 disease-associated microglia (DAM) genes between 5xFAD Syk^{ΔMG} and 5xFAD groups. (G-H) Mouse AKT pathway phosphorylation array conducted on microglia from 5-month-old 5xFAD Syk^{ΔMG} and 5xFAD mice. (G) Representative membranes incubated with 5xFAD and 5xFAD Syk^{ΔMG} microglia measuring 18 different AKT phosphorylation targets. (H) Quantification of dot pixel density normalized with respective positive and negative control sample dot pixel density. Data are plotted in membrane order of phosphorylated protein probes; n of 3 for each group. Statistical significance between experimental groups was calculated by a two-way ANOVA with a Bonferroni post-hoc test (B) and an unpaired Student's t-test (H). *p<0.05, **p<0.01, ****p<0.0001. Error bars represent mean±S.E.M.

FIG. 5A-5L. SYK is critical for microglial uptake and phagocytosis of Aβ (A-F) 5xFAD Syk^{ΔMG} mice and 5xFAD littermate controls received tamoxifen food for 2 weeks starting at 3 weeks of age and then mice were returned to regular food for the remainder of the experiment. Brains were then harvested at 5 months of age to evaluate microglial phagocytosis. (A) Imaris-rendered Aβ plaques (ThioS, pink) and Iba1+ cells (green) with the completely localized Aβ-microglia (engulfed) channel in blue. (B) Percent area of engulfment quantification from a total of ˜20 plaques from 3 matching brain sections per mouse. (C) Imaris-rendered Aβ plaques (ThioS, pink) and CD68 (yellow) with the completely localized Aβ-CD68 (engulfed) channel in blue. (D) Percent area of engulfment quantification from a total of ˜20 plaques from 3 matching brain sections per mouse. (E-F) Mice received intraperitoneal injections of methoxy-X04 (labels fibrillar Aβ) and then microglial phagocytosis of Aβ was determined 3 hours later to evaluate the uptake of methoxy-X04+ labelled Aβ. (E-F) Representative flow cytometry plots and quantification of the percentage of CD11b^{hi}CD45^{int} cells that had taken up methoxy-X04+ labelled Aβ. (G-H) WT and Syk^{fl/fl} LysM^{Cre} bone marrow-derived macrophages (BMDMs) pre-treated with vehicle or 10 μM Tideglusib, a GSK3β inhibitor, for 1 hour prior to treatment with 10 μM CypHer5E-tagged Aβ oligomers. Aβ phagocytosis by BMDMs was determined 24 hours later by measuring CypHer5E fluorescence by flow cytometry. (G-H) Representative flow cytometry plots and quantification of percent CypHer5E CD11b^{hi}F4/80^{hi} cells. (I-L) 10-week-old 5xFAD and 5xFAD Syk^{ΔMG} mice received bilateral intrahippocampal injections of vehicle and CLEC7A agonist pustulan. Seven days post injection (dpi) brains were harvested to measure Aβ load between matched vehicle and pustulan injected hippocampal hemispheres. (J) Representative immunofluorescence staining of D54D2-labeled Aβ (pink) in hippocampal sections. (K-L) Mouse-matched quantification of Aβ in vehicle and pustulan injected hippocampal hemispheres, where each mouse is represented by two dots connected by a line. Statistical significance between experimental groups was calculated by unpaired Student's t-test (B, D, F), one-way ANOVA with multiple comparisons (H), and paired Student's t-test (K-L). *p<0.05, **p<0.01, ***p<0.001, ****p<0.0001. Error bars represent mean±S.E.M. and each data point represents an individual mouse.

FIG. 6A-6J. Syk-deletion in microglia impedes the formation of DAMs in EAE (A-C) Syk^{ΔMG} mice and Syk^{con} littermate controls received tamoxifen food for 2 weeks starting at 3 weeks of age and then mice were returned to regular food for the remainder of the experiment. Mice were then immunized with MOG+CFA and pertussis toxin at 8-14 weeks of age to induce experimental autoimmune encephalomyelitis (EAE). Control mice did not receive EAE induction. (A) Severity of hind-limb paralysis was assessed using a 5-point clinical scoring system. (B-C) Representative images and quantification of spinal cords stained with Luxol fast blue (LFB) to evaluate demyelination. (D-J) Syk^{+/+} Cx3cr1^{ERT2Cre} and Syk^{fl/fl}Cx3cr1^{ERT2Cre} were crossed onto the Ai6-ZsGreen reporter background (denotated as Syk^{con-Ai6} and Syk^{ΔMG-Ai6} mice) to isolate microglia in the EAE disease model. Syk^{con-Ai6} and Syk^{ΔMG-Ai6} mice received tamoxifen food for 2 weeks starting at 3 weeks of age and then mice were returned to regular food for the remainder of the experiment. Mice were then immunized with MOG+CFA and pertussis toxin at 8-14 weeks of age to induce EAE. Spinal cords were harvested from mice on day 35 post-immunization and single-cell RNA-sequencing was performed on FACS-sorted ZsGreen⁺ microglia. (D) Uniform Manifold Approximation and Projection (UMAP) representation of combined Syk^{con-Ai6} and Syk^{ΔMG-Ai6} microglia cell populations. (E) A dot plot representation of cluster defining genes for each cell population. (F) UMAP representation of pseudotime cellular trajectory profiles showing microglia maturation trajectories. (G) UMAP representation of the cell populations present in each of the clusters. (H) Breakdown of cluster proportions. (I) Feature plots depicting several DAM genes. (J) Plotted KEGG and GO terms related to phagocytosis using defining genes of the DAM cluster. Statistical significance between experimental groups was calculated by non-parametric Mann-Whitney U-test (A) and unpaired Student's t-test (C). **p<0.01. Error bars represent mean±S.E.M. and each data point represents an individual mouse (C).

FIG. 7A-7E. Disruption of SYK signaling in microglia during demyelinating disease leads to accumulation of damaged myelin debris and impaired oligodendrogenesis (A-E) Syk^{ΔMG} mice and Syk^{con} littermate controls received tamoxifen food for 2 weeks starting at 3 weeks of age and then mice were returned to regular food for the remainder of the experiment. Adult (8-12-month-old) mice were later fed a diet consisting of 0.3% cuprizone for 5 weeks to induce demyelination. Mice were then either harvested after 5 weeks of cuprizone treatment (demyelination group) or returned to normal chow for one additional week before being harvested (remyelination group). Control mice were not introduced to the cuprizone diet. (A) Representative images of microglia labeled with Iba1 (green) and damaged myelin basic protein (MBP; pink) expression in the corpus callosum. (B) Quantification of dMBP volume in the corpus callosum. (C) Representative images of oligodendrocyte lineage markers in the corpus callosum. (D-E) Quantification of the number of Pdgfra⁺ Olig2⁺ oligodendrocyte precursor cells (D) and number of CC1⁺ Olig2⁺ mature oligodendrocytes (E) in the corpus callosum. Statistical significance between experimental groups was calculated by one-way ANOVA with multiple comparisons (B, D, E). ns=not significant, *p<0.05, **p<0.01, ***p<0.001, ****p<0.0001. Data are mean±S.E.M and combined from two independent experiments and each data point represents an individual mouse.

FIG. 8. Activation of microglia, the brain's residential immune cell, has broadly been shown to exacerbate pathology in many experimental models, as pro-inflammatory responses generated by aberrant innate immune activity trigger unchecked activation of tau kinases and a subsequent cascade involving tau hyperphosphorylation, tau tangle formation, and neuronal dysfunction.

FIG. 9A-9E. Microglial specific SYK deletion dampens hippocampal p-tau burden in 9 mo. male PS19 mice. A) Representative AT8 staining of cortex and hippocampus (CA1). B) Quantification of AT8 MFI calculated by ImageJ. Each point represents an individual image. Tau burden is reduced in the hippocampus with Syk deletion. C-D) p181 Tau ELISA on soluble (C) and insoluble (D) fractions of micro-dissected cortices and hippocampi. Each point represents a mouse. E) SYK deletion tends to improve learning in MWM. Each data point is the average of that group. Error bars represent the SEM, n=5-6 mice.

FIG. 10A-10D. No significant differences observed in synaptic coverage of PS19 SYK^{ΔMG} mice relative to PS19 SYK^{WT} controls. A, C) Representative images of homer (A) and VGLUT1 (C) in both the cortex and hippocampus (CA1). Hot colors represent more intense staining. B, D) Quantification of percent area was calculated with ImageJ. SYK^{ΔMG} seems insignificantly affect homer and VGLUT coverage in the PS19 line. Each data point represents an individual image, n=5-6 mice.

FIG. 11A-11D. Microgliosis is significantly reduced in aged PS19 SYK^{ΔMG} mice. A-B) Representative images of IBA1 and CD68 in both the cortex (A) and hippocampus (B). C-D) PS19 SYK^{ΔMG} mice have significantly decreased IBA1 fluorescence intensity (C) as well as CD68 volume (D). Microglial cell count is unchanged (data not shown). Each data point represents an individual image, n=5-6 mice.

FIG. 12A-12D. Microglial SYK deletion has significant cell non-autonomous effects. A) Representative images of GFAP in both the cortex and hippocampus of PS19 SYK^{WT} and PS19 SYK^{ΔMG} mice. B) Gliosis, measured by GFAP volume, is significantly dampened in the hippocampi of PS19 SYK^{ΔMG} mice. To assess potential cell-cell communication mechanisms, a multiplex was run on micro-dissected cortices and hippocampi. C-D) Quantification of the Bioplex results. Wilcoxon Rank Sum test with Holms' adjustment did not uncover any significantly altered cytokines. Each data point represents an individual mouse. Error bars were omitted due to large variability, n=6.

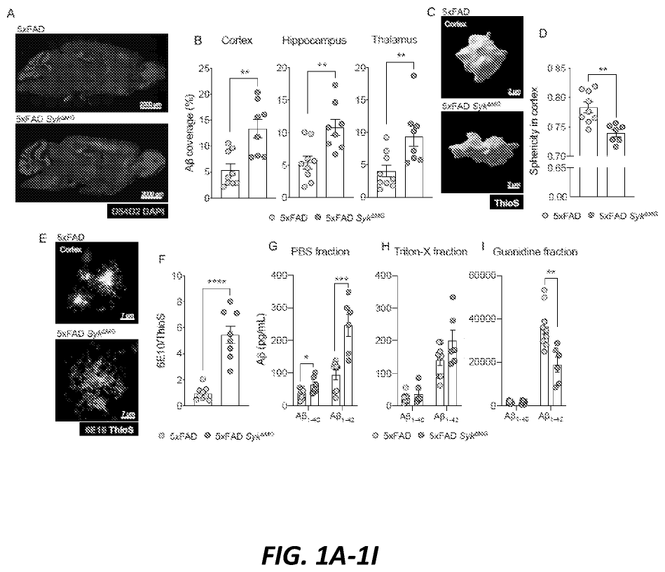

Figure S1A-S1H. Targeted deletion of SYK in tamoxifen-treated Syk^{fl/fl} Cx3cr1^{Ext2Cre} mice, related to FIG. 1 (A-B) Cre-negative 5xFAD Syk^{fl/fl} littermate controls (5xFAD mice) and Cre-negative Syk^{fl/fl} littermate controls (Syk^{con} mice) received tamoxifen food for 2 weeks starting at 3 weeks of age and then mice were returned to regular food for the remainder of the experiment. Spleens and brains were harvested at 5 months of age to evaluate SYK expression in T cells, monocytes, and microglia. T cells, monocytes, and microglia were sorted from single-cell suspensions using respective anti-CD90.2⁺, anti-CD11b⁺, and anti-CD11b+(microglia)-coated magnetic beads and magnetic column sorting. (A) MACS-sorted splenic T cells and monocytes and MACS-sorted brain microglia were harvested from 5-month-old Syk^{con} and 5xFAD mice, and levels of SYK protein (top panel) and Actin or Ponceau-stained protein (bottom panel) were measured by Western blotting. (B) Quantification of intensity of SYK protein bands normalized to total Actin in sorted splenic T cells and monocytes, and intensity of SYK protein bands normalized to Ponceau staining in brain microglia from Syk^{con} and 5xFAD mice. (C-F) Syk^{fl/fl} Cx3cr1^{ERT2Cre} (Syk^{ΔMG} mice) and Cre-negative Syk^{fl/fl} littermate controls (Syk^{con} mice) received tamoxifen food for 2 weeks starting at 3 weeks of age and then mice were returned to regular food for the remainder of the experiment. Brains and spinal cords were later harvested to evaluate SYK deletion. Microglia were sorted from single-cell suspensions using anti-CD11b⁺-coated magnetic beads and magnetic column sorting. (C) Representative flow cytometry gating strategy used to validate purity of MACS-sorted microglia from naïve Syk^{con} and Syk^{ΔMG} combined brain and spinal cord samples. (D) Expression levels of Sykb mRNA from MACS-sorted microglia quantified by qPCR. (E) Levels of SYK protein (top panel) and total protein loaded (bottom panel) from MACS-sorted microglia determined by Western blotting and SDS-PAGE with a stain-free gel, respectively. (F) Quantification of intensity of SYK protein bands normalized to intensity of bands from total protein loaded. (G-H) Syk^{con} and Syk^{ΔMG} mice received tamoxifen food for 2 weeks starting at 3 weeks of age and then mice were returned to regular food for the remainder of the experiment. Spleens were harvested at 5 months of age to evaluate SYK expression in T cells and monocytes. (G) Levels of SYK protein (top panel) and Actin protein (bottom panel) from MACS-sorted splenic T cells and monocytes determined by Western blotting. (H) Quantification of intensity of SYK protein bands normalized to total Actin in sorted splenic T cells and monocytes from Syk^{con} and Syk^{ΔMG} mice. Statistical significance between experimental groups was calculated by unpaired Student's t-test (B, D, F, H). ns=not significant, ***p<0.001, ****p<0.0001. Error bars represent mean±S.E.M. and each data point represents an individual mouse.

Figure S2A-S2X. Microglial SYK deletion in the hippocampus and after disease onset significantly alters AD pathology and microgliosis, however, the loss of SYK does not affect microgliosis in the absence of Aβ, related to FIGS. 1, 2, and 3 (A-G) 5xFAD Syk^{ΔMG} mice and 5xFAD littermate controls received tamoxifen food for 2 weeks starting at 3 weeks of age and then mice were returned to regular food for the remainder of the experiment. Brains were then harvested at 5 months of age to evaluate plaque sphericity and microgliosis in the hippocampus. (A-B) Microglial response to plaques measured by (A) the sphericity of ThioS labeled and Imaris-rendered Aβ plaques in the hippocampus of matched sagittal sections. (B) Quantification of sphericity with 1.00 being the most spherical, combined data from a total of 50-100 plaques from 3 matching brain sections per mouse. (C-E) Microglia were imaged by labeling with Iba1 (green) surrounding Aβ plaques labelled with ThioS (pink) to assess microglial coverage and proximity to plaques. (C) Representative images of Iba1 and ThioS staining in the hippocampus of matching sagittal brain sections. (D) Quantification of microglial numbers in the field of view (FOV) in 40 μm images. (E) Quantification of the number of microglia within a 15 and 30 μm radius surrounding ThioS-labelled Aβ plaques. (F) Representative Imaris rendering of Iba1+microglia in the hippocampus of 5xFAD and 5xFAD Syk^{ΔMG} mice. (G) Sholl analysis quantification from a total of 12 microglia from 3 matching brain sections per mouse (5xFAD n=7, 5xFAD Syk^{ΔMG} n=7). (H-N) 5xFAD Syk^{ΔMG} mice and 5xFAD mice received tamoxifen food for 2 weeks starting at 4 months of age and then mice were returned to regular food for the remainder of the experiment. Brains were then harvested at 8 months of age in this delayed deletion model to evaluate amyloid beta (Aβ) load in the brain. (H-I) Immunofluorescence staining of Aβ (D54D2, red; DAPI, blue) was performed on sagittal sections and the percent area covered by Aβ in the cortex, hippocampus, and thalamus was quantified. (J) Sphericity of ThioflavinS (ThioS)-labeled and Imaris-rendered Aβ plaques in the cortex of matched sagittal sections. (K) Quantification of sphericity with 1.00 being the most spherical, combined data from a total of 50-100 plaques from 3 matching brain sections per mouse. (L-N) Microglia were imaged by labeling with Iba1 (green) surrounding Aβ plaques labelled with ThioS (pink) to assess microglial coverage and proximity to plaques. (L) Representative images of Iba1 and ThioS staining in the cortex of matching sagittal brain sections. (M) Quantification of microglial numbers in the field of view (FOV) in 40 μm images. (N) Quantification of the number of microglia within a 15 and 30 μm radius surrounding ThioS-labelled Aβ plaques. (O-X) 5xFAD Syk^{ΔMG} mice, 5xFAD mice, Syk^{ΔMG} mice, and Syk^{con} mice received tamoxifen food for 2 weeks starting at 3 weeks of age and then mice were returned to regular food for the remainder of the experiment. (O-Q) Mouse body weight was measured at 3 and 6 months of age, while memory and anxiety-related behaviors were evaluated in the Morris water maze (MWM) and elevated plus maze (EPM) at 4 months of age. (O) Mouse body weight recorded in grams (g) across experimental groups. (P) Distance traveled in the MWM on day 4 of the acquisition phase of the test. (Q) Distance traveled in the EPM. (R-V) Brains from Syk^{ΔMG} and Syk^{con} mice were harvested at 5 months of age to evaluate microgliosis in the absence of Aβ. (R) Iba1 (green) and Ki67 (blue) staining was performed on sagittal sections to evaluate Syk^{ΔMG} and Syk^{con} microglial numbers and proliferation. (S) Quantification of microglial numbers in the field of view (FOV) averaged from 3 matching cortical sections per mouse. (T) Quantification of Ki67+ microglia in the field of view (FOV) of the cortex and hippocampus of mice averaged from 3 matching brain sections per mouse. (U) Representative Imaris rendering of Iba1+microglia in the cortex of Syk^{con} and Syk^{ΔMG} mice. (V) Sholl analysis quantification from a total of 12 microglia from 3 matching brain sections per mouse (Syk^{con} n=5, Syk^{ΔMG} n=5). (W-X) Brains were harvested from 5xFAD Syk^{ΔMG} and 5xFAD mice at 5 months of age to evaluate microglial apoptosis by TUNEL staining. (W) 5xFAD Syk^{ΔMG} and 5xFAD microglia labeled with Iba1 (green) and TUNEL (pink) surrounding Aβ plaques. (X) Quantification of TUNEL volume within Iba1⁺ microglia as a measure of apoptosis. Statistical significance between experimental groups was calculated by an unpaired Student's t-test (B, D-E, I, K, M-N, P-Q, S-T, X), two-way ANOVA with a Bonferroni post-hoc test (G, V), and one-way ANOVA with multiple comparisons (O). ns=not significant, *p<0.05, **p<0.01, ***p<0.001. Error bars represent mean±S.E.M.

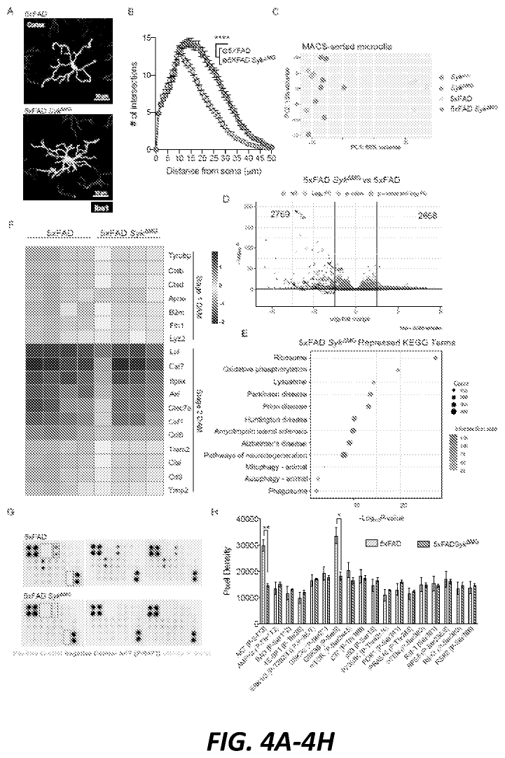

Figure S3A-S3K. Effects of SYK deficiency on microglial gene expression under steady-state conditions and in response to Aβ pathology, related to FIG. 4 (A-K) 5xFAD Syk^{ΔMG}, 5xFAD, Syk^{ΔMG}, and Syk^{con} mice received tamoxifen food for 2 weeks starting at 3 weeks of age and then mice were returned to regular food for the remainder of the experiment. Brains were later harvested at 5 months of age to evaluate microglial activation. (A-D) Microglia from 5-month-old 5xFAD Syk^{ΔMG}, 5xFAD, Syk^{ΔMG}, and Syk^{con} mice were sorted from single-cell brain suspensions using anti-CD11b″-coated magnetic beads and magnetic column sorting, finally, RNA-Seq was performed. (A) Representative flow cytometry gating strategy used to validate purity of MACS-sorted microglia from 5xFAD Syk^{ΔMG}, 5xFAD, Syk^{ΔMG}, and Syk^{con} brain samples. (B) Volcano plots depicting differentially expressed genes (FDR<0.1) where 37 genes are downregulated, and 7 genes are upregulated in Syk^{ΔMG} microglia compared to Syk^{con} microglia. (C) Heatmap representation of the 20 most significantly upregulated and downregulated (FDR<0.1) genes between Syk^{ΔMG} and Syk^{con} mice. (D) Heatmap representation of the 20 most significantly upregulated and downregulated (FDR<0.1) genes between 5xFAD Syk^{ΔMG} and 5xFAD mice. (E-I) Immunohistochemistry validation of RNA-Seq findings in 5-month-old 5xFAD Syk^{ΔMG} and 5xFAD mice. (E) 5xFAD Syk^{ΔMG} and 5xFAD microglia labeled with Iba1 (green) and Tmem119 (pink) surrounding Aβ plaques labelled with ThioS (blue). (F) Quantification of Tmem119 volume colocalized with Iba1. (G) 5xFAD Syk^{ΔMG} and 5xFAD microglia labeled with Iba1 (green) and CLEC7A (pink) surrounding Aβ plaques labelled with ThioS (blue). (H-I) Quantification of total area of CLEC7A surrounding individual Aβ plaques and percent area of CLEC7A normalized to the area of Iba1⁺ cells per Aβ plaque, respectively. Quantification was determined by averaging the Iba1 and CLEC7A area found surrounding ˜30 plaques from 3 matching brain sections per mouse. (J-K) Comparison of the transcriptional changes that arise in microglia with the loss of either SYK or TREM2 in 5xFAD mice. 5xFAD Syk^{ΔMG} and 5xFAD brains were harvested at 5 months of age and their microglia were sorted from single-cell brain suspensions using anti-CD11b⁺-coated magnetic beads and magnetic column sorting to perform RNA-Seq. 8-month-old 5xFAD Trem2^{-/-} and littermate 5xFAD microglia were FACS-sorted and analyzed by RNA-Seq in (Griciuc et al. 2019). (J) Venn diagram of significantly upregulated and downregulated genes in 5xFAD Trem2^{-/-} and 5xFAD Syk^{ΔMG} microglia compared with their respective littermate 5xFAD controls (FD<0.05). (K) Molecular function (MF) term enrichment scatter plot highlighting major functions that are repressed in 5xFAD Trem2^{-/-} and 5xFAD Syk^{ΔMG} microglia in comparison to littermate 5xFAD microglia. Statistical significance between experimental groups was calculated by unpaired Student's t-test (F, H-I). *p<0.05, ***p<0.001. Error bars represent mean±S.E.M. and each data point represents an individual mouse.

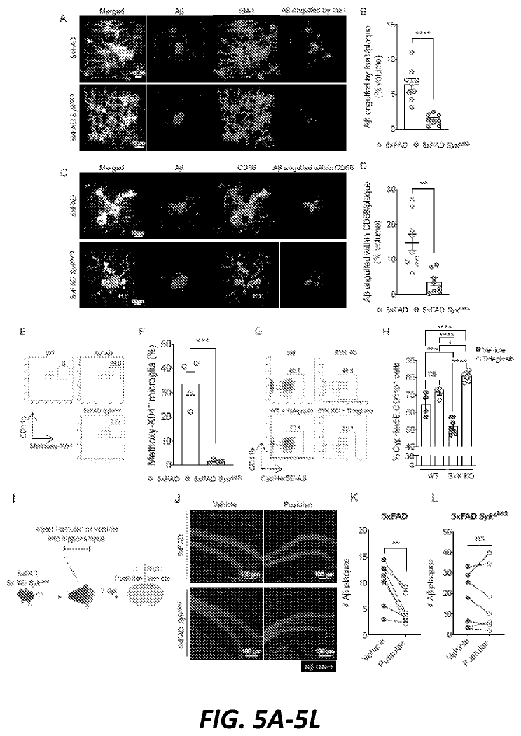

Figure S4A-S4F. Genetic ablation of SYK in microglia leads to increased levels of microglial lipid droplet accumulation and ROS production in 5xFAD mice, related to FIG. 5 (A-D) 5xFAD Syk^{ΔMG} mice and 5xFAD littermate controls received tamoxifen food for 2 weeks starting at 4 months of age and then mice were returned to regular food for the remainder of the experiment. Microglia from 8-month-old 5xFAD Syk^{ΔMG} and 5xFAD mice were sorted from single-cell brain suspensions using flow-cytometry. (A) Representative histograms of BODIPY-labeled lipid-droplet accumulation in CD11b CD45^{int} microglia. (B) Mean Fluorescence Intensity (MFI) quantification of BODIPY in 5xFAD and 5xFAD Syk^{ΔMG} microglia. (C) Representative histograms of CellROX-labeled reactive oxygen species (ROS) in CD11b⁺CD45^{int} microglia. (D) MFI quantification of CellROX in 5xFAD and 5xFAD Syk^{ΔMG} microglia. (E-F) 5xFAD Syk^{ΔMG} mice and Cre-negative 5xFAD mice received tamoxifen food for 2 weeks starting at 3 weeks of age and then mice were returned to regular food for the remainder of the experiment. Brains were later harvested and Iba1 staining was performed at 5 months of age to evaluate microglial activation. (E) 5xFAD Syk^{ΔMG} and 5xFAD microglia labeled with Iba1 (green) and CD68 (blue) surrounding Aβ plaques labelled with ThioS (pink). (F) Volumetric quantification of CD68 normalized to Iba1 volume. Statistical significance between experimental groups was calculated by unpaired Student's t-test (B, D, F). ns=not significant, *p<0.05. Error bars represent mean±S.E.M. and each data point represents an individual mouse.

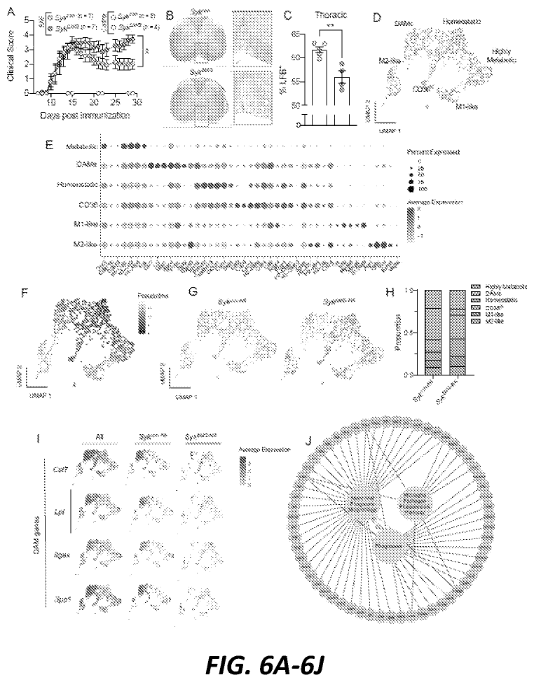

Figure S5A-S5I. Syk^{ΔMG} mice have increased numbers of cytokine producing T cells infiltrating the CNS but have modestly reduced peripheral T cell responses during EAE, related to FIG. 6 (A-H) Syk^{ΔMG} mice and Syk^{con} littermate controls received tamoxifen food for 2 weeks starting at 3 weeks of age and then mice were returned to regular food for the remainder of the experiment. Mice were later immunized with MOG+CFA and pertussis toxin at 8-14 weeks of age to induce experimental autoimmune encephalomyelitis (EAE). (A-E) Spinal cords were then harvested from mice during the effector phase of clinical disease (at least 30 days post-immunization) to evaluate immune cell responses by flow cytometry. (A) Representative flow cytometry gating strategy. (B) Quantification of immune cell populations. (C) Representative dot plots of effector cytokine producing CD4⁺ T cells after no stimulation or 5-hour ex vivo stimulation with PMA and ionomycin in the presence of monensin. (D) Quantification of the frequencies of effector cytokine producing CD4⁺ T cells. (E) Quantification of the total numbers of effector cytokine producing-CD4⁺ T cells. (F-H) Spleens were harvested from mice during the effector phase of clinical disease (at least 30 days post-immunization) to evaluate immune cell responses by flow cytometry. (F) Representative dot plots of EAE effector-cytokine producing CD4⁺ T cells from EAE effector phase spleens after no stimulation or 5-hour ex vivo stimulation with PMA and ionomycin in the presence of monensin. (G) Quantification of effector cytokine production by CD4⁺ T cells. Data are combined from 2 independent experiments. (H) Quantification of secreted cytokine levels in culture media from Syk^{con} and Syk^{ΔMG} EAE effector phase splenocytes stimulated ex vivo with MOG₃₅₋₅₅ peptide for 48 hours. Levels of indicated analytes were measured by multiplex cytokine assay. (I) Flow cytometry-based sorting of ZsGreen+ microglia from Syk^{ΔMG-Ai6} and Syk^{con-Ai6} mice. Syk^{ΔMG} and Syk^{con} mice were crossed onto the Ai6-ZsGreen reporter background (denotated as Syk^{ΔMG-Ai6} and Syk^{con-Ai6} mice) to isolate microglia in the EAE disease model. Spinal cords were harvested from mice on day 35 post-immunization and single-cell RNA-sequencing was performed on FACS-sorted ZsGreen⁺ microglia. Statistical significance between experimental groups was calculated by unpaired Student's t-test (B, D-E, G-H). *p<0.05, **p<0.01. Error bars represent mean±S.E.M. and each data point represents an individual mouse.

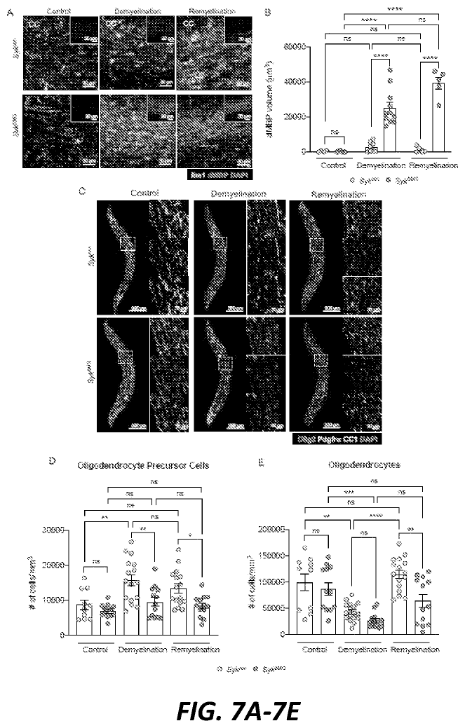

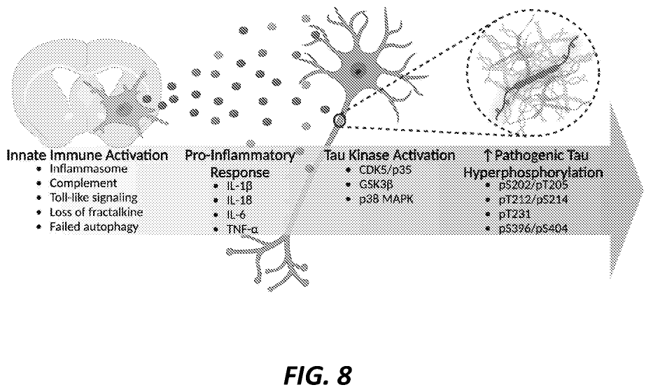

Figure S6A-S6J. Disruption of SYK signaling in microglia during demyelinating disease leads to impaired microglial response and reduced OPC proliferation, related to FIG. 7 (A-B) Syk^{ΔMG} mice and Syk^{con} littermate controls received tamoxifen food for 2 weeks starting at 3 weeks of age and then mice were returned to regular food for the remainder of the experiment. Mice were harvest at 8 months of age and total myelin levels were evaluated in the corpus callosum. (A) Syk^{ΔMG} mice and Syk^{con} corpus callosum labeled with myelin basic protein (MBP; green) and DAPI (blue). (B) Quantification of percent area covered by MBP in the corpus callosum at baseline. (C-J) Syk^{ΔMG} mice and Syk^{con} mice received tamoxifen food for 2 weeks starting at 3 weeks of age and then mice were returned to regular food for the remainder of the experiment. Adult (8-12-month-old) mice were later fed a diet consisting of 0.3% cuprizone for 5 weeks to induce demyelination. Mice were then either harvested after 5 weeks of cuprizone treatment (demyelination group) or returned to normal chow for one additional week before being harvesting (remyelination group). Control mice were not introduced to the cuprizone diet. (C-D) Representative images and quantification of the number of Iba1⁺ cells (green) in the corpus callosum. (E-F) Representative images and quantification of microglial apoptosis measured by TUNEL⁺ volume (pink) in Iba1⁺ microglia (green) in the corpus callosum. (G) Representative images of Iba1⁺ microglia (green) and CD68 (pink) expression in the corpus callosum. (H) Volume of CD68 colocalized to the volume of Iba1 in the corpus callosum. (I) Representative images of proliferating Ki67⁺ (pink) Pdgfra⁺ Olig2⁺ (blue; green) oligodendrocyte precursor cells (OPCs) in the corpus callosum. (J) Quantification of Ki67⁺ OPCs in the corpus callosum. Statistical significance between experimental groups was calculated by unpaired Student's t-test (B) and one-way ANOVA with multiple comparisons (D, F, H, J). ns=not significant, *p<0.05, **p<0.01, ***p<0.001, ****p<0.0001. Data are mean±S.E.M and combined from two independent experiments and each data point represents an individual mouse.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th ed}., J. Wiley & Sons (New York, NY 2013); and Sambrook and Russel, Molecular Cloning: A Laboratory Manual 4^{th ed}., Cold Spring Harbor Laboratory Press (Cold Spring Harbor, NY 2012), provide one skilled in the art with a general guide to many of the terms used in the present application.

Abbreviations: Aβ, amyloid beta; AD, Alzheimer's disease; Border-associated macrophages, (BAMs); CLEC, C-type lectin; CNS, central nervous system; con, control; DAM, disease-associated microglia; DAMPs, damage-associated molecular patterns; EAE, expeirmental autoimmune encephalomyelitis; EPM, elevated plus maze; FDR, false discovery rate; GSK3β, glycogen synthase kinase 3 beta; GWAS, genome-wide association studies; intraperitoneal, i.p.; ITAM, immunoreceptor tyrosine-based activation motif; ITIM, immunoreceptor tyrosine-based inhibition motif; LDAM, lipid-droplet-accumulating microglia; MG, microglia; MGnDs, neurodegenerative disease-associated microglia; MWM, morris water maze; OPC, olidendrocyte progenitor cell; PC, principal component; RNA-seq, RNA-sequencing; scRNA-seq, single-cell RNA-sequencing; SYK, spleen tyrosine kinase; ThioS, Thioflavin S.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations. “Plurality” means at least two.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.” As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The terms “patient,” “individual,” or “subject” are used interchangeably herein, and refer to a mammal, particularly, a human. The patient may have mild, intermediate or severe disease. The patient may be an individual, at risk of developing a disease, in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters, and primates. Mammals include, but are not limited to, humans, farm animals, sport animals and pets.

As used herein, “health care provider” includes either an individual or an institution that provides preventive, curative, promotional or rehabilitative health care services to a subject, such as a patient. In one embodiment, the data is provided to a health care provider so that they may use it in their diagnosis/treatment of the patient.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

A “compound,” as used herein, refers to any type of substance or agent that is commonly considered a drug, or a candidate for use as a drug, as well as combinations and mixtures of the above.

As used herein, the term “pharmaceutically-acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

The term “gene” refers to a nucleic acid sequence that comprises control and coding sequences necessary for producing a polypeptide or precursor. The polypeptide may be encoded by a full-length coding sequence or by any portion of the coding sequence. A gene may contain one or more modifications in either the coding or the untranslated regions that could affect the biological activity or the chemical structure of the expression product, the rate of expression, or the manner of expression control. Such modifications include, but are not limited to, methylation, mutations, insertions, deletions, and substitutions of one or more nucleotides. The gene may constitute an uninterrupted coding sequence, or it may include one or more introns, bound by the appropriate splice junctions.

The term “gene expression” refers to the process by which a nucleic acid sequence undergoes successful transcription and/or translation such that detectable levels of the nucleotide sequence are expressed.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. Furthermore, the term “nucleic acid sequences” contemplates the complementary sequence and specifically includes any nucleic acid sequence that is substantially homologous to the both the nucleic acid sequence and its complement.

The term “activation” as used herein refers to any alteration of a signaling pathway or biological response including, for example, increases above basal levels, restoration to basal levels from an inhibited state, and stimulation of the pathway above basal levels.

The term “inhibition” as used herein refers to any alteration of a signaling pathway or biological response including, for example, decreases below basal levels, restoration to basal levels from an activated state, and inhibition of the pathway above basal levels.

The term “differential expression” refers to both quantitative as well as qualitative differences in the temporal and tissue expression patterns of a gene in diseased tissues or cells versus normal adjacent tissue. For example, a differentially expressed gene may have its expression activated or partially or completely inactivated in normal versus disease conditions or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions but is not detectable in both. Stated another way, a gene is differentially expressed when expression of the gene occurs at a higher or lower level in the diseased tissues or cells of a patient relative to the level of its expression in the normal (disease-free) tissues or cells of the patient and/or control tissues or cells.

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of neurodegeneration. Moreover, a sample obtained from a patient can be divided and only a portion may be used for diagnosis. Further, the sample, or a portion thereof, can be stored under conditions to maintain sample for later analysis. The definition specifically encompasses blood, and other liquid samples of biological origin (including, but not limited to, peripheral blood, serum, plasma, urine, saliva and synovial fluid), solid tissue samples such as a biopsy specimen or tissue cultures or cells derived therefrom and the progeny thereof. Samples may be collected as part of routine physician visits, e.g., at the doctor's office. The definition also includes samples that have been manipulated in any way after their procurement, such as by centrifugation, filtration, precipitation, dialysis, chromatography, treatment with reagents, washed, or enriched for certain cell or DNA populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a liquid sample, cell, organ, or patient, e.g., a control or normal cell, organ, or patient, exhibiting, for example, normal traits.

### I. SYK

Tyrosine-protein kinase SYK, also known as spleen tyrosine kinase, is an enzyme which in humans is encoded by the SYK gene. The human protein sequence for SYK is provide below (SEQ ID NO: 1) and found at protein accession number P43405 (NCBI Gene ID-6850).

MASSGMADSANHLPFFFGNITREEAEDYLVQGGMSDGLYLLRQSRNYLG
GFALSVAHGRKAHHYTIERELNGTYAIAGGRTHASPADLCHYHSQESDG
LVCLLKKPFNRPQGVQPKTGPFEDLKENLIREYVKQTWNLQGQALEQAI
ISQKPQLEKLIATTAHEKMPWFHGKISREESEQIVLIGSKTNGKFLIRA
RDNNGSYALCLLHEGKVLHYRIDKDKTGKLSIPEGKKFDTLWQLVEHYS
YKADGLLRVLTVPCQKIGTQGNVNFGGRPQLPGSHPATWSAGGIISRIK
SYSFPKPGHRKSSPAQGNRQESTVSFNPYEPELAPWAADKGPQREALPM
DTEVYESPYADPEEIRPKEVYLDRKLLTLEDKELGSGNFGTVKKGYYQM
KKVVKTVAVKILKNEANDPALKDELLAEANVMQQLDNPYIVRMIGICEA
ESWMLVMEMAELGPLNKYLQQNRHVKDKNIIELVHQVSMGMKYLEESNF
VHRDLAARNVLLVTQHYAKISDFGLSKALRADENYYKAQTHGKWPVKWY
APECINYYKFSSKSDVWSFGVLMWEAFSYGQKPYRGMKGSEVTAMLEKG
ERMGCPAGCPREMYDLMNLCWTYDVENRPGFAAVELRLRNYYYDVVN

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion. “Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity, such as about 75%, about 80%, about 85%, about 90%, about 95%, about 96%, about 97%, about 98%, about 99% about 100% between two sequences can be determined using techniques similar to those described above, with or without allowing gaps. In calculating percent identity, typically exact matches are counted.

As used herein, a “substantially homologous amino acid sequences” includes those amino acid sequences which have at least about 90% homology, at least about 95% homology, at least about 96% homology, at least about 97% homology, at least about 98% homology, or at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

### II. Disease/Disorders

Provided herein are method to prevent or treat a neurodegenerative disease.

In some aspects, the neurodegenerative disease is Aβ-driven neurodegeneration, such as Alzheimer's Disease (AD).

In other aspects, the neurodegenerative disease is a demyelinating disease (any condition that causes damage to the protective covering (myelin sheath) that surrounds nerve fibers), such as multiple sclerosis (MS), Balo's disease, acute-disseminated encephalomyelitis, progressive multifocal leukoencephalopathy, Charcot-Marie-Tooth Disease, Guillain-Barre Syndrome (GBS), HTLV-I Associated Myelopathy (HAM), Neuromyelitis Optica (Devic's Disease), Schilder's Disease, Transverse Myelitis, and extrapontine myelinolysis.

In other aspects, the neurodegenerative disease is a tauopathy. Tauopathies are neurodegenerative disorders characterized by the deposition of abnormal tau protein in the brain, these include Pick disease, progressive supranuclear palsy, corticobasal degeneration, argyrophilic grain disease, frontotemporal lobar degeneration (FTLD-Tau), Alzheimer disease (AD; considered a secondary tauopathy), chronic traumatic encephalopathy, post-encephalitic parkinsonism and/or Parkinsonism linked to chromosome 17.

### III. Activators/Inhibitors of SYK

### A. Activators of SYK/Treatment of Amyloid Beta-Mediated and Demyelinating Neurological Diseases

In certain embodiments the methods comprise administering a neurodegeneration therapy. Such therapies include the actions of the physician or clinician. For example, if a physician makes a diagnosis or prognosis of neurodegeneration, then a certain regime would follow.

Provided herein are agents and compositions thereof that activate SYK. In one aspect the agent is a β-D-glucan. In another aspect, the agent is pustulan. The chemical structure of Pustulan is:

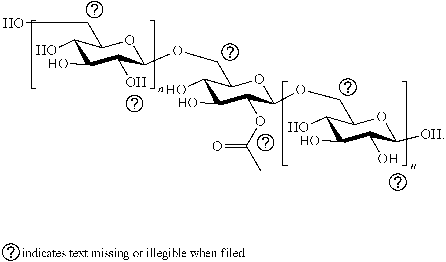

In another aspect, the agent is an anti-CLEC7A agonist antibody. In some aspects, the agent is IL-2.

### B. Inhibitors of SYK/Treatment of Tauopathies

In certain embodiments the methods comprise administering a neurodegeneration therapy. Such therapies include the actions of the physician or clinician. For example, if a physician makes a diagnosis or prognosis of neurodegeneration, then a certain regime would follow.

Provided herein are agents and compositions thereof that inhibit SYK. In one aspect the agent is GSK 143. The chemical structure of GSK 143 is:

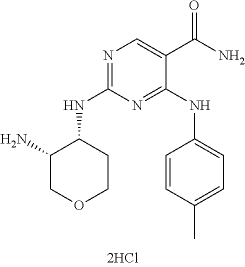 [SMILES: Cc1ccc(Nc2nc(N[C@@H]3CCOC[C@@H]3N)ncc2C(N)=O)cc1]

(2-[[(3R,4R)-3-Aminotetrahydro-2H-pyran-4-yl]amino]-4-[(4-methylphenyl)amino]-5-pyrimidinecarboxamide dihydrochloride).

In one aspect the agent is Gusacitinib. The chemical structure of Gusacitinib is:

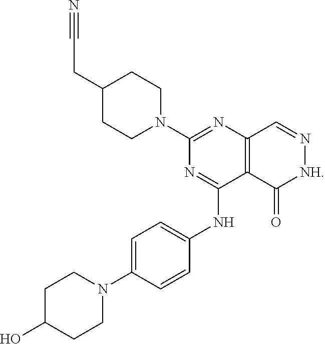 [SMILES: N#CCC1CCN(c2nc(Nc3ccc(N4CCC(O)CC4)cc3)c3c(=O)[nH]ncc3n2)CC1]

In one aspect the agent is R406. The chemical structure of R406 is:

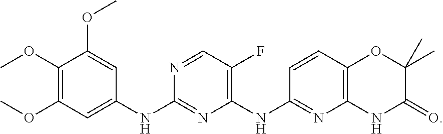

In one aspect the agent is Fostamatinib, including Fostamatinib disodium and Fostamatinib disodium hexahydrate. The chemical structure of Fostamatinib is:

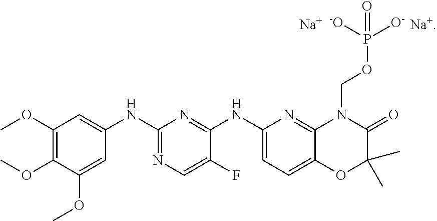

In one aspect the agent is Piceatannol. The chemical structure of Piceatannol is:

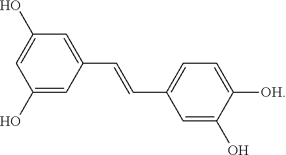 [SMILES: Oc1cc(O)cc(/C=C/c2ccc(O)c(O)c2)c1]

In one aspect the agent is Dehydroabietic acid. The chemical structure of Dehydroabietic acid is:

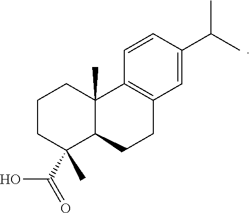 [SMILES: CC(C)c1ccc2c(c1)CC[C@H]1[C@](C)(C(=O)O)CCC[C@]21C]

In one aspect the agent is Cerdulatinib, including Cerdulaltinib hydrochloride. The chemical structure of Cerdulatinib is:

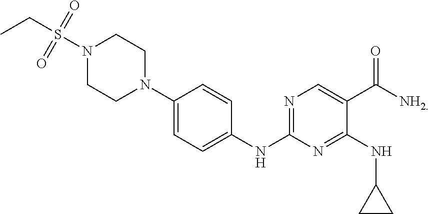 [SMILES: CCS(=O)(=O)N1CCN(c2ccc(Nc3ncc(C(N)=O)c(NC4CC4)n3)cc2)CC1]

In one aspect the agent is MNS (3,4-Methylenedioxy-β-nitrostyrene). The chemical structure of MNS (3,4-Methylenedioxy-β-nitrostyrene) is:

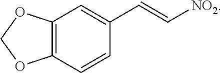 [SMILES: O=[N+]([O-])/C=C/c1ccc2c(c1)OCO2]

In one aspect the agent is R112. The chemical structure of R112 is:

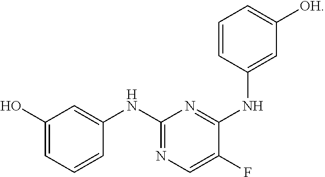 [SMILES: Oc1cccc(Nc2ncc(F)c(Nc3cccc(O)c3)n2)c1]

In one aspect the agent is BAY-61-3606. The chemical structure of BAY-61-3606 is:

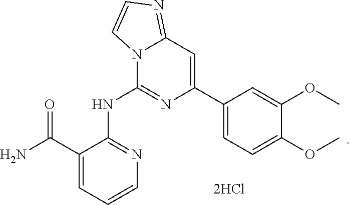 [SMILES: COc1ccc(-c2cc3nccn3c(Nc3ncccc3C(N)=O)n2)cc1OC]

In one aspect the agent is RO9021. The chemical structure of RO9021 is:

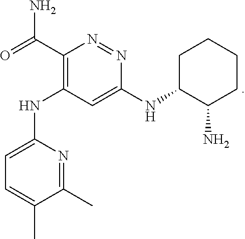 [SMILES: Cc1ccc(Nc2cc(N[C@@H]3CCCC[C@@H]3N)nnc2C(N)=O)nc1C]

In one aspect the agent is Entospletinib (GS-9973). The chemical structure of Entospletinib (GS-9973) is:

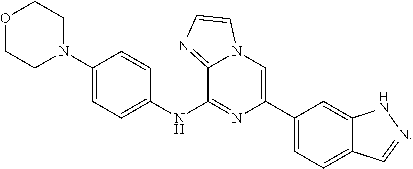 [SMILES: c1cn2cc(-c3ccc4cn[nH]c4c3)nc(Nc3ccc(N4CCOCC4)cc3)c2n1]

In one aspect the agent is PRT-060318 2HCl. The chemical structure of PRT-060318 2HCl is:

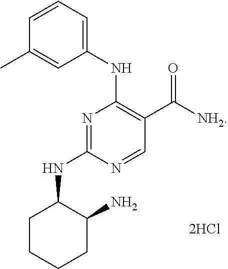 [SMILES: Cc1cccc(Nc2nc(N[C@@H]3CCCC[C@@H]3N)ncc2C(N)=O)c1]

In one aspect the agent is PRT062607 (P505-15) HCl. The chemical structure of PRT062607 (P505-15) HCl is:

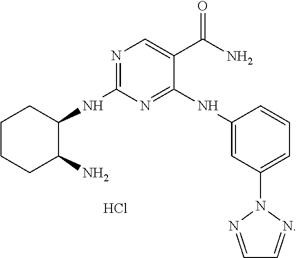 [SMILES: NC(=O)c1cnc(N[C@@H]2CCCC[C@@H]2N)nc1Nc1cccc(-n2nccn2)c1 ; Cl]

In one aspect the agent is TAK-659. The chemical structure of TAK-659 is:

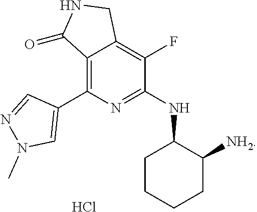 [SMILES: Cn1cc(-c2nc(N[C@@H]3CCCC[C@@H]3N)c(F)c3c2C(=O)NC3)cn1 ; Cl]

In one aspect the agent is SRX3207. The chemical structure of SRX3207 is:

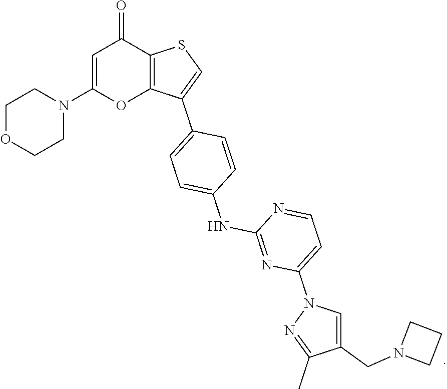 [SMILES: Cc1nn(-c2ccnc(Nc3ccc(-c4csc5c(=O)cc(N6CCOCC6)oc45)cc3)n2)cc1CN1CCC1]

In one aspect the agent is Lanraplenib (GS-9876). The chemical structure of Lanraplenib (GS-9876) is:

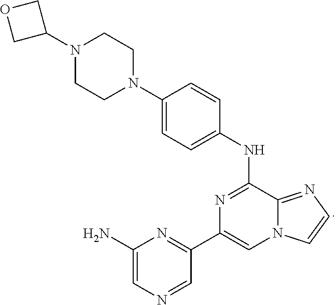 [SMILES: Nc1cncc(-c2cn3ccnc3c(Nc3ccc(N4CCN(C5COC5)CC4)cc3)n2)n1]

Or salts of any agents provided herein.

### IV. Treatment/Administration

Agents/compounds described herein can be administered by any art available means, including orally and/or by injection.

The present disclosure also contemplates pharmaceutical compositions comprising one or more agents/compounds to activate or inhibit SYK, one mor more compounds/agents disclosed herein, one or more pharmaceutically acceptable carriers, diluents, excipients or combinations thereof. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, isotonic and absorption delaying agents that are physiologically compatible. The carrier is suitable for, among other applications, parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time release formulation, for example in a composition that includes a slow-release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

The compounds can be administered by a variety of dosage forms as known in the art. Any biologically acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition can vary according to factors such as the disease state, age, gender, weight, patient history, risk factors, predisposition to disease, administration route, pre-existing treatment regime (e.g., possible interactions with other medications), and weight of the individual. Dosage regimens can be adjusted to provide the optimum therapeutic response. For example, a single bolus can be administered, several divided doses can be administered over time, or the dose can be proportionally reduced or increased as indicated by the exigencies of therapeutic situation.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and can be directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds disclosed herein or an appropriate pharmaceutical composition thereof are effective, the compounds disclosed herein can be administered in an effective amount. The dosages as suitable for this disclosure can be a composition, a pharmaceutical composition or any other compositions described herein.

The dosage can be administered once, twice, or thrice a day, although more frequent dosing intervals are possible. The dosage can be administered every day, every 2 days, every 3 days, every 4 days, every 5 days, every 6 days, and/or every 7 days (once a week). The dosage can be administered daily for up to and including 30 days, preferably between 7-10 days. Or the dosage can be administered twice a day for 10 days. If the patient requires treatment for a chronic disease or condition, the dosage can be administered for as long as signs and/or symptoms persist. The patient may require “maintenance treatment” where the patient is receiving dosages every day for months, years, or the remainder of their lives. In addition, the composition can affect prophylaxis of recurring symptoms. For example, the dosage can be administered once or twice a day to prevent the onset of symptoms in patients at risk, especially for asymptomatic patients.

The compositions described herein can be administered in any of the following routes: buccal, epicutaneous, epidural, infusion, inhalation, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

The term “therapeutically effective amount” as used herein, refers to that amount of one or more compounds that elicits a biological or medicinal response in a tissue system, animal or human, that is being sought by a researcher, veterinarian, medical doctor or other clinician, which includes alleviation of the symptoms of the disease or disorder being treated. The therapeutically effective amount can be that which may treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The present disclosure also contemplates compounds having a % activation of at least about 10%, at least about 20%, at least about 30%, at least about 40%, at least about 50%, at least about 60%, at least about 70%, at least about 80%, at least about 90%, at least about 99%; or about 20% to about 100%, about 30% to about 90%, about 40% to about 95%, about 50% to about 90% or about 70% to about 95%.

Without further elaboration, it is believed that one skilled in the art, using the preceding description, can utilize the present invention to the fullest extent. The following example is illustrative only, and not limiting of the remainder of the disclosure in any way whatsoever.

### V. EXAMPLES

The following example is put forth so as to provide those of ordinary skill in the art with a complete disclosure and description of how the compounds, compositions, articles, devices, and/or methods described and claimed herein are made and evaluated and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example I

### Introduction

Neurodegenerative disorders, such as Alzheimer's disease (AD), are major public health issues that are likely to increase in prevalence with the aging population. In general terms, neurodegenerative diseases are thought to be driven by the accumulation of neurotoxic material such as amyloid beta (Aβ) or myelin debris in the central nervous system (CNS) (Nussbaum and Ellis, 2003; Trapp and Nave, 2008). The buildup of neurotoxic agents is believed to cause neuronal damage and death, which can ultimately lead to various forms of neurological dysfunction that include cognitive decline, motor abnormalities, mental disorders, and loss of inhibition (Chung et al., 2018; Taylor et al., 2002; Vickers et al., 2009). Mounting evidence suggests that microglia, which are the professional phagocytes of the CNS, are critically involved in ensuring the proper containment and removal of neurotoxic material in neurodegenerative disease pathogenesis (Condello et al., 2015; Hickman et al., 2018; Lampron et al., 2015). Indeed, human genome-wide association studies (GWAS) have implicated mutations in microglial receptors in the development of several neurodegenerative diseases (Cooper-Knock et al., 2017; Efthymiou and Goate, 2017; IMSG, 2019).

Most notably, emerging evidence from both AD patients and neurodegenerative mouse models has identified roles for TREM2, CD33, and CD22 in disease progression (Bemiller et al., 2017; Krasemann et al., 2017; Malik et al., 2013; Pluvinage et al., 2019; Ulland et al., 2017; Wang et al., 2015). While there is growing interest in targeting these receptors to treat neurodegenerative disease, there is a lack of knowledge of the major downstream signaling molecules and effector mechanisms employed by these receptors to influence disease pathogenesis.

Provided herein, is whether the intracellular signaling molecule, spleen tyrosine kinase (SYK), is involved in coordinating neuroprotective functions in microglia during neurodegenerative disease. SYK is perhaps best known for the role that it plays in mounting protective antifungal immune responses downstream of C-type lectin (CLEC) receptors expressed on innate immune cells (Lionakis et al., 2017; Mocsai et al., 2010). In addition to orchestrating immune responses during fungal infections, SYK has been identified as the central kinase that instructs signaling and effector functions downstream of ITAM (immunoreceptor tyrosine-based activation motif)- and ITIM (immunoreceptor tyrosine-based inhibition motif)-containing receptors (Estus et al., 2019; Kimura et al., 2014; Konishi and Kiyama, 2018; Lumb et al., 2016). More specifically, SYK activation downstream of ITAM-containing receptor engagement often leads to the induction of inflammatory cytokine production and phagocytosis (Abram and Lowell, 2007; Underhill and Goodridge, 2007). In contrast, stimulation of ITIM-containing receptors activates the protein tyrosine phosphatase SHP-1 which acts as a brake on SYK-dependent signaling (Abram and Lowell, 2007; Underhill and Goodridge, 2007). Given that TREM2, CD33, and CD22 are ITAM/ITIM-containing receptors, and that SYK activation is often seen in microglia that surround Aβ and other forms of neurotoxic material (Schweig et al., 2017), it was of interest to delineate whether SYK is a regulator of microglial responses in neurodegenerative disease. While the ability of SYK to modulate Aβ and tau biology has been explored in previous in vitro studies using immortalized CNS lines and pharmacological agents with well-described off-target effects (Lawlor et al., 2018; Paris et al., 2014), the extent to which SYK influences in vivo microglial responses and neurodegenerative disease pathogenesis currently remains poorly understood.

Here, it is shown that microglia-specific deletion of SYK leads to elevated levels of Aβ deposition, exacerbated neuropathology, and cognitive impairment in the 5xFAD mouse model of AD. It is further demonstrated that SYK is involved in both the compaction and phagocytosis of Aβ by microglia as well as the regulation AKT/GSK3β-signaling. Recent studies have shown that microglia transition from a homeostatic state to a disease-associated microglia (DAM) phenotype during neurodegenerative disease, and it is widely thought that this transformation is required for microglia to exert their optimal neuroprotective functions in degenerative disease (Keren-Shaul et al., 2017). Interestingly, SYK is identified as a key intracellular regulator of DAM phenotype acquisition and it is further shown that CLEC7A-induced activation of SYK in 5xFAD mice promotes improved clearance of Aβ. Moreover, it is shown that the neuroprotective effects of SYK on microglia are not confined to Aβ-driven models of degenerative disease, but rather that DAM induction and microglial phagocytosis are also dependent on SYK signaling in the context of demyelinating disease. Collectively, these findings define SYK as a central regulator of neuroprotective microglial responses in neurodegenerative disease.

### Experimental Model and Subject Details

### Mice

All mouse experiments were performed in accordance with the relevant guidelines and regulations of the University of Virginia and approved by the University of Virginia Animal Care and Use Committee. 5xFAD mice (Stock #34848-JAX), Syk^{fl/fl} mice (Stock #017309) and Cx3cr1^{ERT2cre} mice (Stock #020940) were obtained from The Jackson Laboratory and were crossed to generate Syk^{fl/fl} (denoted as Syk^{con}), Syk^{fl/fl} Cx3cr1^{ERT2cre} (denoted as Syk^{ΔMG}), 5xFAD Syk^{fl/fl} (denoted as 5xFAD), and 5xFAD Syk^{fl/fl} Cx3cr1^{ERT2cre} (denoted as 5xFAD Syk^{ΔMG}) experimental mice. Upon weaning, female Syk^{con} and Syk^{ΔMG}, 5xFAD, and 5xFAD Syk^{ΔMG} littermates were fed tamoxifen diet (Envigo Teklad #TD.130858) ad libitum for two weeks and then returned to normal chow. Ai6-ZsGreen (Stock #007906-JAX) reporter mice were generously provided by Dr. Tajie Harris and crossed with Syk^{+/+} Cx3cr1^{ERT2Cre} and Syk^{fl/fl}Cx3cr1^{ERT2Cre} mice. In EAE experiments, Syk^{con}, Syk^{ΔMG}, Syk^{con-Ai6}, and Syk^{ΔMG-Ai6} mice were fed tamoxifen diet for two weeks upon weaning and then returned to normal chow. In cuprizone experiments, Syk^{con} and Syk^{ΔMG} were fed tamoxifen diet for two weeks upon weaning and then returned to normal chow. Mice were housed for AD and demyelinating experiments in specific pathogen-free conditions under standard 12-hour light/dark cycle conditions in rooms equipped with control for temperature (21±1.5° C.) and humidity (50±10%).

### Experimental Autoimmune Encephalomyelitis (EAE) Induction and Scoring

On the day of immunization (Day 0), mice were injected subcutaneously over each shoulder with 100 μl of an emulsion containing 0.5 mg/ml MOG₃₅₋₅₅ peptide (Bio-Synthesis, 12668-01) and 1.25 mg/ml heat-killed Mycobacterium tuberculosis (Becton, Dickinson, & Company, 231141) in Complete Freund's Adjuvant (Sigma Aldrich, F5881). Mice were intraperitoneally injected with 200 ng of Pertussis toxin (List Biological Laboratories, 180) on days 0 and 2 post-immunization.

Beginning at approximately day 7 post-immunization, mice were monitored daily for onset of hind-limb paralysis and scored for EAE severity using the following 5-point clinical scoring system: 0=normal tail; 0.5=limp at tip of tail; 1=completely limp tail; 1.5=partial hind limb weakness/mouse can be flipped onto its back; 2-complete hindlimb weakness with abnormal gait; 3-partial hind-limb paralysis; 3.5=complete hind-limb paralysis in both legs; 4=hind- and fore-limb paralysis; 5=moribund/dead.

### Cuprizone Model

For the cuprizone model, adult mice were fed regular chow mixed with 0.3% cuprizone (Sigma, 14690) ad libitum for 5 weeks to induce demyelination.

### Method Details

### Western Blotting

MACS-sorted microglia were resuspended in Western blot lysis buffer [dH₂O, RIPA, complete Protease Inhibitor Cocktail (Roche), and PhosSTOP (Roche)]. Protein concentration was measured using Pierce 660 nm Protein Assay Reagent (Thermo Scientific, 22660). 4×SDS loading dye was added to protein lysates and incubated at 95° C. for 3 minutes. For each sample, 25 μg of protein was loaded per lane of a 4-20% Mini-PROTEAN TGX Stain-Free Protein Gel (BioRad, 4568093) and run at 120 volts for 1.5 hours using Mini-PROTEAN Tetra Cell (BioRad, 1658004). Proteins were transferred to an Immun-Blot Low Fluorescence PVDF membrane (BioRad, 1620264) using Trans-Blot Turbo transfer system (BioRad, 1704150) set to 2 mini gels and mixed MW for 21 minutes. SYK protein was probed using anti-SYK (D3Z1E) XP Rabbit mAb (Cell Signaling Technologies, 13198, 1:1000 overnight at 4° C.) and goat anti-rabbit IgG StarBright Blue 700 secondary antibody (BioRad, 12004161, 1:1000 for 2 hours at room temperature). The stain-free gel and blotted membrane were imaged using ChemiDoc MP Imaging System (BioRad, 12003154). Total protein loaded was quantified using Image lab touch software (BioRad), Beta-Actin (Cell Signaling, 12620), Ponceau S stain (Sigma-Aldrich, P7170) and SYK protein were quantified using Fiji. Samples underwent the same preparation for the AKT pathway phosphorylation kit (RayBiotech, AAH-AKT-1-8). The AKT assay was performed in accordance with manual instructions and pixel density was analyzed using Adobe Photoshop.

### Brain Sample Preparation

Mice were euthanized using CO₂ asphyxiation and transcardially perfused with 20 ml of 1×PBS. For AD experiments, brains were dissected out with the left hemisphere drop-fixed in 4% paraformaldehyde over night at 4° C. and the right hemisphere flash-frozen and stored at −80° C. Drop-fixed samples were transferred to 30% sucrose for 48 hours and then mounted and frozen in Tissue-Plus OCT compound (Fisher Scientific). These brains were then sectioned at 50 μm in thickness using a cryostat (Leica) and stored in PBS+0.05% sodium azide at 4° C. for downstream staining and imaging. The flash-frozen brains were thawed for RNA and protein extraction and mechanically homogenized in 500 μl of Tissue Protein Extraction Reagent T-PER (Thermo Fisher, 78510) containing phosphatase inhibitor cocktail PhosSTOP (Roche, 04906845001) and protease inhibitor cocktail complete (Roche, 11873580001). Following homogenization, 50 μl of the brain sample was diluted in 500 μl Trizol for future RNA extraction and stored at −80° C. The stock brain samples were then spun down at 16,000 rpm for 10 minutes and the supernatant and pellet were isolated for soluble and insoluble amyloid beta analyses, respectively. For EAE experiments, brain tissue was dissected and immersion fixed in 4% paraformaldehyde for 48 hours, followed by dehydration in 30% sucrose and freezing in OCT. Free-floating cryosections were cut (40 μm) and collected in PBS containing 0.02% sodium azide and stored at 4° C. until further analysis using the methods outlined above.

### ELISA

Brain samples underwent guanidine-extraction in which pelleted brain samples were incubated 1:6 in 5 M guanidine HCL/50 mM tris, pH=8.0 at room temperature for 3 hours, then diluted 1:5 in PBS containing protease inhibitor cocktail complete (Roche, 11873580001), centrifuged at 16,000 g for 20 minutes at 4° C., and the supernatant was collected and stored at −80° C. pending ELISA. Triton-X extraction was performed by diluting the pelleted brain samples 1:5 in 1% Triton-X-100 in T-PER buffer and sonicating the samples for 30 minutes at room temperature, then spun down at 16,000 g for 20 minutes at 4° C. and the supernatant stored at −80° C. until used for Aβ measurement by ELISA. Amyloid beta 40 or 42 Mouse ELISA kits were utilized (Thermo Fisher, KMB3481, KMB3441) on samples obtained with the soluble fraction (T-PER extracted supernatant) diluted 1:10, Triton-X fraction diluted 1:40, and the guanidine fraction diluted 1:200 following manufacturer's instructions.

### Immunofluorescence Microscopy

Floating brains sectioned stored in PBS+0.05% sodium azide were blocked with 2% donkey serum, 1% bovine serum albumin, 0.1% triton, 0.05% tween in PBS for 1 hour at room temperature before applying the primary antibody master mix diluted in this block overnight at 4° C. Samples were stained with anti-AB (D54D2, Cell Signaling, 1:300; or 6e10, BioLegend, 1:1000) to label plaques. To study microglial morphology and numbers, sections were stained with Iba1 (ab5076, Abcam, 1:300). To further characterize microglia, we labeled with Ki67-EF660 (SoIA15, Thermo Fisher, 1:100), anti-Clec7a (R1-8g7, Invivogen, 1:30), Tmem119 (ab209064, Abcam, 1:300), and anti-CD68 (FA-11, BioRad, 1:1000). Neuronal health was probed by staining for anti-APP (Y188, ab32136, Abcam, 1:750), anti-phospho-tau (AT8, Thermo Fisher, 1:500), and anti-NeuN (MAB377, Millipore Sigma, 1:500). For cuprizone experiments, sections were stained with PDGFRα (AF1062, R&D Systems, 1:200), CC1 (OP80, 1:200, Millipore), Olig2 (Aβ9610, 1:500, Millipore), dMBP (Aβ5864, Millipore Sigma, 1:2000), and MBP (ab7349, Abcam, 1:1000). Sections were then washed 3 times for 10 minutes at room temperature in PBS and 0.05% tween-20, then incubated in matched donkey Alexa Fluor 488, 594, 647 anti-rabbit, -goat, -rat, -streptavidin, and -mouse (Thermo Fisher, 1:1000 dilution) at room temperature for 2 hours. Samples were washed again 3 times for 10 minutes at room temperature and incubated with DAPI (1:1000) for 10 minutes at room temperature or stained for plaques with ThioflavinS (Sigma-Aldrich, 2 mg/10 ml) for 8 minutes followed by three 2-minute washes with 50% ethanol at room temperature. To investigate cell death, sections were stained by TUNEL (Millipore Sigma, 11684795910) following the manufacturer's protocol. All tissue sections were then transferred to wells containing PBS before being mounted to glass slides with ProLongGold antifade reagent (Invitrogen, P36930) and coverslips. Mounted slides were stored at 4° C. until imaged using LAS AF software (Leica Microsystems) on a Leica TCS SP8 confocal microscope. Quantification of images were performed using Fiji software or Imaris software (9.5.1).

### Behavior

All behavior experiments were performed between 8 am and 12 μm in a blinded fashion. All mice were 4 months old at the time of the assay. Mice were transported from their home vivarium room to the behavior core and allowed 30 minutes to habituate before beginning each test.

### Morris Water Maze

The MWM was done as described previously (Da Mesquita et al., 2018). In brief, the test involved four days of training consisting of four trials, one day of probe consisting of one trial, and two days of reversal consisting of four trials per mouse. Mice were alternately placed facing different visual cues for each trial in a 23° C. pool made opaque with white paint. The hidden platform was placed 1 cm below the water surface. Each trail lasted 60 seconds, and the mouse was placed on the hidden platform for 5 seconds if unable to locate it within the 60 second trial. All trials were tracked and scored using video tracking software (Noldus Ethovision XT).

### Elevated Plus Maze

EPM was used to investigate anxiety in mice and was performed as described previously (Lammert et al., 2020). The maze has two open arms (35×6 cm²) and two closed arms (35×6 cm²) with 20 cm-tall black plexiglass walls elevated 121 cm from the floor. The mice were placed in the center square connecting the open and closed arms and allowed to explore during a 5-minute trial. Activity was monitored and scored using video tracking software (Noldus Ethovision XT).

### In Vivo Aβ Phagocytosis Assay

5xFAD, 5xFAD Syk^{ΔMG}, and littermate controls were intraperitoneally injected with 10 mg/kg methoxy-X04 (ApexBio, B5769) in a 1:1 ratio of PBS and DMSO. A brain harvest was completed 3 hours after injection in which mice were euthanized using CO₂ asphyxiation before being intracardially perfused with 20 mL of PBS+Na heparin (5 units/mL). The brains were placed in a Hanks buffer saline solution (HBSS) (Thermo Fisher, 14025092) with DNAse I (50 U/mL) (Sigma-Aldrich, 10104159001) and papain (2 mg/mL) (Worthington, LS003124) and homogenized using a 10 mL serological pipette. The brain homogenates were then incubated at 37° C. for 15 minutes. This process was repeated for a total of 3 times with the final two homogenizations accomplished using a 5 mL serological pipette. The brain homogenates were then passed through a 70 μm strainer to create a single-cell suspension. The cell suspension was then placed in 20 mL of DMEM/F12 buffer (21331020, Thermo Fisher) with 10% fetal bovine serum (FBS) (Thermo Fisher, 10082147), 1% Anti-anti (Thermo Fisher, 15240096), and 1% Glutamax (Thermo Fisher, 35050061) and spun down with a slow brake at 300 g for 10 minutes. The cell pellet was then resuspended in 13 mL of 37% Percoll (Cytvia, 17-0891-02) and spun down with no brake at 2000 rpm for 12 minutes. Myelin was removed and cells were resuspended in MACS buffer (Miltenyi Biotec, 130-0910376) to wash. Flow cytometry for microglia was then performed (as described below) with additional gating for methoxy-X04

### CypHer5E-Aβ Preparation

Monomerization of Aβ (1-42) (641-15, California peptide) was achieved using a previously published protocol (Stine et al., 2011), using hexafluoroisopropanol (HFIP) (52517, Sigma-Aldrich). 5 mM monomeric Aβ samples were incubated for 24 hours at 4° C. in F12 media to make a 200 μM stock of oligomeric Aβ. Samples were then incubated with CypHer5E-NHS ester (PA15401, Cytvia) diluted in 0.1 M sodium bicarbonate for 30 minutes covered and at room temperature. Following incubation, Biospin columns (7326227, Bio-Rad) were used to quench unbound dye. CypHer5E-tagged Aβ oligomers were stored at 4° C. prior to BMDM treatment.

### In Vitro Aβ Phagocytosis Assay

Bone marrow-derived macrophages (BMDMs) were isolated from the hindlimbs of WT and Syk^{fl/fl} LysM^{Cre} mice. Marrow-containing bones were sprayed with 70% ethanol before being placed in IMDM (Thermo Fisher, 12440-053) containing penicillin/streptomycin (P/S) (Thermo Fisher, 15140163). A 25-gauge needle was used to flush marrow from the bones using 20 ml of IMDM containing P/S. An 18-gauge needle was then used to triturate flushed bone marrow 5 times to make a single-cell suspension. Samples were spun down at 1500 rpm for 5 minutes at 4° C. Cell pellets were resuspended BMDM media containing IMDM, 10% FBS, 1% non-essential amino acids, 1% P/S, and 50 ng/ml M-CSF (PeproTech, 315-02). Cell plating was performed using 150×25 mm culture dishes (430597, Thomas Scientific). After three days, 5 ml of BMDM media was added to each dish. Six days post initial cell plating, media was aspirated from dishes and 10 ml of PBS was added to each plate and incubated for 10 minutes at 4° C. BMDMs were removed from the dish using a scraper and transferred to a conical tube, spun down, and resuspended in BMDM media. 100,000 cells/well were pipetted in into a flat bottom 96 well plate. The next day, BMDMs were treated with vehicle or 10 μM of Tideglusib (Selleck Chemicals, S2823) 1 hour prior to treatment with 10 μM of oligomeric Aβ tagged with CypHer5E. 24 hours post Aβ treatment, BMDMs were then collected, and flow cytometry was used to assess CypHer5E fluorescence.

### Intrahippocampal Injection

5xFAD and 5xFAD Syk^{ΔMG} mice were anesthetized before receiving a bilateral hippocampal injection of 2 μl of vehicle or 2 μg pustulan into the right and left hemisphere of the hippocampus (at +2 mm lateral, −2 mm posterior, and −2 mm ventral relative to the intersection of the coronal and sagittal suture (bregma) at a rate of 200 nl/min) using a stereotaxic frame (51730U, Stoelting) and nanoliter injector (NL2010MC2T, World Precision Instruments). Seven days post injection, mice were euthanized using CO₂ and transcardially perfused before preparing brains for immunofluorescent staining to evaluate Aβ clearance in the hippocampus. Images were analyzed using Fiji software and Imaris software (9.5.1).

### MACS Isolation of Microglia, T Cells, and Monocytes

Mice were euthanized with CO₂ and immediately transcardially perfused with 20 ml 1×PBS. For AD experiments, brains were collected, and their meninges and choroid plexus removed then the MACS-sorting protocol was used to isolate microglia as described (Norris et al., 2018). For EAE experiments, spinal cords were dissected and kept on ice in DMEM (Thermo Fisher, 11885-084) with penicillin/streptomycin (Thermo Fisher, 15140163). Tissues were homogenized by gently mashing through a 70 μm cell strainer. Homogenates were centrifuged at 1500 rpm for 5 minutes then resuspended in 13 ml 37% isotonic Percoll (GE Healthcare, 17-0891-01) in 1×PBS. Samples were centrifuged at 2000 rpm for 12 minutes at room temperature with no brake. After centrifugation, the top myelin layer and supernatant were aspirated and the cell pellet was resuspended in MACS buffer (Miltenyi Biotec, 130-0910376) to proceed with column purification of microglia using CD11b microbeads (Miltenyi, 130-093-634), purification of T cells using CD90.2 microbeads (Miltenyi, 130-121,278), and purification of monocytes using CD11b microbeads (Miltenyi, 130-049-601). We then performed sorting utilizing LS columns and a QuadroMACS magnet (Miltenyi, 130-042-401 and 130-091-051) according to manufacturer's instructions. Column-bound cells were analyzed for purity by flow cytometry, probed for SYK expression by qPCR and Western blotting, or submitted for RNA-seq.

### Flow Cytometry

For MACS-sort validation, an aliquot of the microglia-positive and -negative fractions were transferred to a 96-well round bottom plate, then washed with 1×PBS and spun down at 1600 rpm for 5 minutes. The cells were then stained with fixable viability dye (eBioscience, 65-0866-14) at 1:1000 for 30 minutes at 4° C. Following incubation, cells were then washed with FACS buffer (pH 7.4; 0.1 M PBS; 1 mM EDTA, and 1% BSA). Cells were then stained 1:200 with CD11b (APC), CD45 (PE-Cy7), and TCR β chain (Brilliant Violet 510) flow antibodies (all from eBioscience) in FACS buffer for 15 minutes at 4° C. The cell pellets were then washed with FACS buffer then resuspended in 100 μl of FACS buffer. Microglia were identified as the CD45^{int} and CD11b^{hi} after gating for single and live cells.

For lipid-droplet-accumulation and reactive oxygen species (ROS) assessment in microglia, mice were euthanized with CO₂ at 8 months of age and immediately transcardially perfused with 20 ml 1×PBS. Brains were collected and their meninges and choroid plexus removed and prepped as a single cell suspension as described in (Norris et al., 2018). Brain homogenates were centrifuged at 1500 rpm for 5 minutes then resuspended in 13 ml 37% isotonic Percoll (GE Healthcare, 17-0891-01) in 1×PBS. Samples were centrifuged at 2000 rpm for 12 minutes at room temperature with no brake. After centrifugation, the top myelin layer and supernatant were aspirated, and the cell pellet was washed with PBS. Cells were then stained either 1:500 with CellROX (Thermo Fisher, C10491) for 30 minutes or 1:2000 with BODIPY (Invitrogen, D3861) for 10 minutes diluted in PBS at 37° C. Cells were spun down and washed with FACS buffer (pH 7.4; 0.1 M PBS; 1 mM EDTA, and 1% BSA). Cells were then stained 1:200 with CD11b (APC), CD45 (PE-Cy7), and TCR β chain (Brilliant Violet 510) flow antibodies (all from eBioscience) in FACS buffer for 15 minutes at 4° C. The cell pellets were then washed with FACS buffer then resuspended 1:5000 with DAPI in 100 μl of FACS buffer. Microglia were identified as the CD45^{int} and CD11b^{hi} after gating for single and live cells.

For flow cytometry staining of BMDMs, cells were washed with FACS buffer and centrifuged at 1500 rpm for 5 min and resuspended in 100 μl of FACS buffer with fluorescently labeled antibodies (all from eBioscience) specific for CD11b (clone M1/70) and F4/80 (clone BM8) diluted 1:200. Cells were incubated in the dark for 20 minutes at room temperature, washed with FACS buffer, and resuspended 1:5000 with DAPI in 100 μl of FACS buffer. BMDMs were identified as the CD11b^{hi} and F4/80^{hi} after gating for single and live cells.

For flow cytometry staining in EAE experiments, cells were plated (100 μl of resuspended spinal cord or 1×10⁶ splenocytes) in a 96-well plate and washed with FACS buffer. After centrifugation at 1500 rpm for 5 minutes and removal of supernatants, cells were resuspended in 100 μl 1×PBS with 1:1000 fixable viability dye (eBioscience, 65-0866-14) and 1:1000 Fc Block (eBioscience, 14-0161-86). Cells were incubated at 4° C. for 30 min. Cells were then washed with FACS buffer, centrifuged at 1500 rpm for 5 min, and resuspended in 100 μl of FACS buffer with fluorescently labeled antibodies (all from eBioscience) specific for CD45 (clone 30-F11), CD11b (clone M1/70), Gr-1 (clone RB6-8C5), MH-CII (clone M5/114.15.2), TCRβ (clone H57-597), CD4 (clone RM4-5), CD8 (clone 53-6.7), CD11c (clone N418), and CD80 (clone 16-10A1) diluted 1:200. Cells were incubated in the dark for 20 minutes at room temperature, washed twice with FACS buffer, and fixed with 1% paraformaldehyde in FACS buffer.

For intracellular cytokine staining, cells were plated (100 μl of resuspended spinal cord or draining lymph node cells or 1×10⁶ splenocytes) in a 96-well plate in IMDM stimulation media (Iscove's Modified Dulbecco's Media) (Thermo Fisher, 12440-053), penicillin/streptomycin (Thermo Fisher, 15140163), 10% heat-inactivated fetal bovine serum (Thermo Fisher, 10082147), 1% L-glutamine (Thermo Fisher, 25030-081), and 50 μM beta-mercaptoethanol (Thermo Fisher, 21985-023)] with 20 ng/ml PMA (Sigma-Aldrich, P1585), 1 μg/ml ionomycin (Sigma-Aldrich, I9657), and 1:1000 monensin (eBioscience, 00-4505-51). Cells were incubated for 5 hours at 37° C. with 5% CO₂, then washed with 1×PBS prior to proceeding with surface staining for flow cytometry as described above. Cells were fixed and permeabilized using IC fixation buffer (eBioscience, 00-8222-49) and permeabilization buffer (eBioscience, 00-8333-56) following manufacturer's instructions. Cells were then stained with 100 μl fluorescently labeled antibodies (all from Thermo Fisher) for GM-CSF (clone MP1-22E9), IFN-γ (clone XMG1.2), and IL-17A (clone eBio17B7) diluted 1:200 in 1× permeabilization buffer for 20 min at room temperature. Cells were washed twice with 1× permeabilization buffer, then twice with FACS buffer.

Sample data were acquired within a few days of fixation using a Gallios flow cytometer (10 colors, 3 lasers, B5-R1-V2 Configuration with Kaluza Acquisition; Beckman Coulter) and analyzed using FlowJo software (Becton, Dickinson, & Company).

### Multiplex Cytokine Assay

Immune cells isolated from spinal cords or spleens were plated in a 96-well plate at up to 2×10⁵ cells/well and stimulated with 30 g/ml MOG₃₅₋₅₅ peptide in IMDM stimulation media for 48 hours at 37° C. with 5% CO₂. After incubation, cells were centrifuged at 1600 rpm for 5 minutes and supernatants were collected for storage at −80° C.

Supernatants were assayed for concentrations of various cytokines using Bio-Rad Bio-Plex Pro reagent kit (Bio-Rad, 171-304070M) and Bio-Plex Pro Mouse Cytokine 23-Plex Group I magnetic beads and detection antibodies for IL-la, IL-1β, IL-2, IL-4, IL-6, IL-17A, G-CSF, GM-CSF, IFN-γ, KC, and TNF-α according to manufacturers' instructions (Bio-Rad). Sample data for Bio-Plex Pro assays were acquired with a Bio-Plex 200 System (Bio-Rad) and analyzed using Bio-Plex Manager software (Bio-Rad).

### Histopathological Analysis of EAE Spinal Cords

Mice were euthanized with CO₂ and immediately transcardially perfused with 20 ml 1×PBS followed by 20 ml 10% neutral buffered formalin (NBF; Azer Scientific). Spinal columns were dissected, and immersion fixed in 10% NBF for at least 48 hours. Spinal columns were cut into cervical, thoracic, and lumbar pieces. Spinal cords from each piece were carefully dissected from the column, embedded in paraffin, sectioned coronally from the rostral end of each piece, and mounted on slides. Sections were stained with Luxol Fast Blue (LFB; Thermo Fisher, 212170250) to label myelin. In brief, after deparaffinization, sections were incubated at 60° C. for 16-24 hours in 0.1% Luxol Fast Blue in 95% ethanol+0.05% acetic acid. Excess stain was removed with 95% ethanol and slides were dipped in 0.05% lithium carbonate (Thermo Fisher, 446322500) in dH₂O for 10-20 seconds then in 70% ethanol to remove LFB staining from gray matter but not from myelinated white matter. Slides were washed with dH₂O then counterstained with hematoxylin (Sigma Aldrich, HHS128) to label nuclei. Brightfield images were acquired on a Keyence BZ-X810 microscope at 4× and 40× magnification.

### Isolation of Immune Cells

Mice were euthanized with CO₂ and immediately transcardially perfused with 20 ml 1×PBS. Spinal cords and spleens were dissected and kept on ice in DMEM (Thermo Fisher, 11885-084) with penicillin/streptomycin (Thermo Fisher, 15140163). Tissues were homogenized by gently mashing through a 70 μm cell strainer. For spinal cords, homogenates were centrifuged at 1500 rpm for 5 minutes then resuspended in 13 ml 37% isotonic Percoll (GE Healthcare, 17-0891-01) in 1×PBS. Samples were centrifuged at 2000 rpm for 12 minutes at room temperature with no brake. After centrifugation, the top myelin layer and supernatant were aspirated, and the cell pellet was resuspended in 500 μl DMEM with penicillin/streptomycin. For spleens, homogenates were centrifuged at 1500 rpm for 5 minutes then resuspended in 2 ml ACK lysis buffer (Quality Biological, 118-156-101) and incubated at room temperature for 3 minutes to lyse red blood cells. Cells were washed and resuspended in DMEM with penicillin/streptomycin. Cells were counted using a Cellometer Auto 2000 (Nexelcom Bioscience).

RNA Isolation, cDNA Synthesis, qPCR

RNA was isolated from the left hemisphere of the brain of 5xFAD and 5xFAD Syk^{ΔMG} mice. 50 μl of brain homogenate (described in the brain sample preparation section) was added to 500 μl of TRIzol (Life Technologies, 15596018). Following vortexing of these samples, 200 μl of chloroform (Fisher Scientific, BP1145-1) was added and incubated for 5 minutes before being spun down at 14,000 rpm at 4° C. for 15 minutes. The top aqueous fraction was transferred to a new tube and an equal volume of isopropanol (Sigma-Aldrich, 19516) was added then vortexed. The samples were incubated at room temperature for 10 minutes and then spun down at 12,000 rpm at 4° C. for 5 minutes. The pellet was then washed with 1 mL 70% ethanol 2 times, then allowed to air dry for 15 minutes before resuspending the RNA pellet in 100 μl of DNAse/RNAse free water. RNA was isolated from MACS-sorted microglia in the spinal cord using an RNeasy Micro kit (Qiagen, 74004) according to manufacturer's instructions. Sample quality and quantity for AD and EAE samples was evaluated using NanoDrop 2000 Spectrophotometer (Thermo Fisher). The RNA was then converted to cDNA using a Sensifast cDNA Synthesis kit (Bioline, BIO-65054). Levels of Sykb (Mm01333035_m1) and Gapdh (Mm99999915_g1) mRNA were determined using Taqman Gene Expression Assay primer/probe mix (Thermo Fisher), Sensifast Probe No-ROX kit (Bioline, BIO-86005), and a CFX384 Real-Time PCR System (BioRad, 1855484). All reagents were used according to manufacturer's instructions.

### FACS Sorting for RNA Sequencing

Ai6-ZsGreen reporter mice possess a LoxP-flanked STOP cassette that prevents the expression of green fluorescent protein variant ZsGreen1 until the stop site is excised after tamoxifen induction of Cre-recombinase activity. Following the withdrawal of tamoxifen, short-lived Ai6-ZsGreen expressing cells in the periphery are promptly replaced by newly derived cells that lack Ai6-ZsGreen expression. In contrast, the long-lived nature of microglia allows them retain their Ai6-ZsGreen signal for months post-tamoxifen withdrawal (Goldmann et al., 2013). Syk^{con-Ai6} mice were euthanized on EAE Day 35 with CO₂ and immediately transcardially perfused with 20 ml ice cold 1×PBS. Spinal cords were dissected and placed on ice in DMEM+10% FBS. Tissues were gently homogenized by mashing through a cell strainer then centrifuged at 1500 rpm for 5 min. Pellets were resuspended in 13 ml 37% isotonic Percoll (GE Healthcare, 17-0891-01) in 1×PBS. Samples were centrifuged at 2000 rpm for 12 minutes at room temperature with no brake. After centrifugation, the top myelin layer and supernatant were aspirated, and the cell pellet was resuspended in FACS buffer. Cell suspensions were incubated with Fc Block and antibodies for CD45, CD11b, and Gr-1. After two washes with FACS buffer, cells were resuspended in FACS buffer+0.2 mg/ml DAPI and sorted on DAPI⁻ CD45⁺ ZsGreen⁺ cells using an Influx Cell Sorter (BD) in the University of Virginia Flow Cytometry Core Facility.

### RNA Sequencing Data Analysis

### AD Microglia RNA-Seq

MACS-sorted microglia were sent to GENEWIZ Next Generation Sequencing. The raw sequencing reads (FASTQ files) were aligned to the UCSC mm10 mouse genome build using the splice-aware read aligner HISAT2. Samtools was used for quality control filtering. Reads were sorted into feature counts with HTSeq. DESeq2 (v1.32.0) was used to normalize the raw counts based on read depth, perform principal component analysis, and conduct differential expression analysis. The p-values were corrected with the Benjamini-Hochberg procedure to limit false positives arising from multiple testing. The significantly repressed and enhanced genes were put into GProfiler to gather the KEGG terms. The analysis itself was performed using the Seq2Pathway, fgsea, tidyverse, and dplyr software packages. Heatmaps were generated using the pheatmap R package [https://github.com/raivokolde/pheatmap] while other plots were made with the lattice (http://lattice.r-forge.r-project.org/) or ggplot2 [https://ggplot2.tidyverse.org] packages.

EAE Microglia scRNA-Seq

FACS-sorted single cell suspensions were submitted to the University of Virginia Genome Analysis and Technology Core for single-cell RNA sequencing library preparation. The raw sequencing reads (FASTQ files) were aligned to the UCSC mm10 mouse genome build using Cell Ranger (v1.3.1) which performs alignment, filtering, barcode counting and unique molecular identifier (UMI) counting. R studio (v4.0.5) was used for all downstream analyses and Seurat (v.4.0.2) was used for filtering out low-quality cells, normalization of the data, determination of cluster defining markers and graphing of the data on UMAP. Low-quality cells were excluded in an initial quality-control (QC) step by removing genes expressed in fewer than three cells, cells with fewer than 150 genes expressed, and cells expressing more than 5000 genes. Cells with more than 20% of mitochondrial-associated genes and cells with more than 5% hemoglobin among their expressed genes were also removed. Genes with high variance were selected using the FindVariableGenes function, then the dimensionality of the data was reduced by principal component analysis (PCA) and identified by random sampling significant principal components (PCs) for each sample with the PCElbowPlot function. Cells were clustered with Seurat's FindClusters function. Differential gene expression analysis was performed within clusters using the ZinBWave function and DESeq2 (v1.32.0). ToppCluster (Cincinnati Children's) was used for network analyses to identify KEGG and GO terms in the DAM cluster. Data was organized and graphs were created using patchwork, dplyr, tidyverse, and Seurat. The frequency plot was created using Prism GraphPad. Pseudotime analysis was conducted using Monocle (v0.2.3.0).

### Statistics

Mean values, SEM values, Student's t test (unpaired), and one-way ANOVA were calculated using Prism software (GraphPad). Significance for pooled EAE experiments was performed by a Mann-Whitney test. P values less than 0.05 were considered significant. *p<0.05, **p<0.01, ***p<0.001, ****p<0.0001.

### Results

### SYK Signaling in Microglia Limits Aβ Accumulation

To interrogate how SYK signaling in microglia impacts Aβ-mediated neurodegenerative disease, Syk^{fl/fl} Cx3cr1^{ERT2Cre} mice (hereafter referred to as Syk^{ΔMG} mice) were generated as a genetic tool to delete SYK from microglia. Syk^{ΔMG} mice were then crossed with 5xFAD mice which is an AD mouse model that develops aggressive Aβ pathology starting at 1.5 months of age (Richard et al., 2015). SYK expression is unchanged between 5xFAD and non-5xFAD immune cells that have been described to modulate AD pathogenesis (Figures S1A and S1B) (Chen et al., 2020; Gate et al., 2020; Hickman et al., 2018; Machhi et al., 2021; Munawara et al., 2021). 5xFAD Syk^{ΔMG} mice were given tamoxifen food for 2 weeks after weaning to induce deletion of SYK and then returned to normal chow to allow for peripheral Cx3cr1-expressing immune cells to turn over and regain Syk expression, while permitting long-lived microglia to remain SYK-deficient (Figures S1C-S1H). As controls, Cre-negative Syk^{fl/fl} 5xFAD littermates (hereafter referred to as 5xFAD mice) were similarly fed tamoxifen for 2 weeks at weaning and then returned to normal chow for the remainder of the experiment. It is of note that this genetic targeting strategy may also induce deletion of SYK in Cx3cr1-expressing CNS border-associated macrophages (BAMs), which do not undergo the frequent turnover characteristic of Cx3cr1-expressing peripheral immune cells (Wu et al., 2021). Therefore, SYK deletion in Syk^{ΔMG} mice is likely not limited to microglia found in the brain parenchyma but may also encompass self-renewing BAMs found in CNS border regions such as the meninges and choroid plexus (Van Hove et al., 2019).

Using these newly generated transgenic mouse lines, it was found that SYK-deletion in 5xFAD Syk^{ΔMG} mice leads to significantly elevated accumulation of Aβ in the cortex, hippocampus, and thalamus at 5 months of age (FIGS. 1A and 1B). Microglia help to limit the spread of pathological Aβ in the brain parenchyma by forming a barrier around Aβ deposits and promoting the physical compaction of Aβ into dense spherical plaques, which ultimately decreases Aβ interaction with susceptible neurons (Condello et al., 2015). Therefore, the lack of Aβ plaque sphericity is often used to identify potentially neurotoxic Aβ aggregates and to provide insights into the efficacy of microglial compaction (Condello et al., 2018; Wang et al., 2016). In addition to higher amounts of Aβ load, it was observed that Aβ plaques in the cortex and hippocampus of 5xFAD Syk^{ΔMG} mice exhibited lower sphericity than the plaques found in 5xFAD littermate controls (FIGS. 1C, 1D, S2A, S2B). Delayed deletion of SYK in 4-month-old 5xFAD mice yields similarly increased plaque load in the hippocampus and decreased plaque sphericity in the cortex when harvested at 8 months of age (Figure S2H-S2K). This suggests that microglial SYK remains influential in attenuating pathology after disease-onset in 5xFAD mice. In further support of there being less efficient compaction of Aβ into plaques by SYK-deficient microglia, it was observed that Aβ aggregates in 5xFAD Syk^{ΔMG} mice were more filamentous (increased 6E10 antibody labeling that detects filamentous Aβ (Lee et al., 2018)) and less compact (decreased staining of Thioflavin S (ThioS) that detects inert plaques (Lee et al., 2018)) than the Aβ deposits found in 5xFAD littermate controls (FIGS. 1E and 1F). Taken together, these findings suggest that SYK signaling in microglia plays a role in the control of Aβ accumulation and compaction.

Microglia are required for the consolidation of soluble Aβ oligomers into insoluble fibrils and the construction of these fibrils into compact plaques (Brown et al., 2020; Huang et al., 2021; Martin et al., 1994). Aβ40 and Aβ42 are the predominant isoforms that make up plaques, with soluble Aβ42 being one of the most highly pathological forms of Aβ, especially in comparison to the more inert insoluble forms of Aβ42 (Ferreira et al., 2015; Gu and Guo, 2013; Klein et al., 1999). To evaluate whether the absence of SYK in microglia also affects the levels of both soluble and insoluble Aβ in 5xFAD mice, Aβ load was evaluated using an ELISA and various extraction techniques to isolate Aβ based on solubility. From these studies, it was observed that the levels of soluble Aβ40 and Aβ42 were considerably higher in 5xFAD Syk 1. (mice than in 5xFAD littermate controls (FIG. 1G). Although no appreciable differences in Aβ ELISA levels were detected between experimental groups in the Triton X-100 extraction samples, reduced levels of insoluble Aβ42 was observed in the guanidine fractions isolated from 5xFAD Syk^{ΔMG} mice (FIGS. 1H and 1I), indicating that SYK centrally contributes to the ability of microglia to construct more inert Aβ structures. Altogether, these data indicate that SYK is needed for Aβ consolidation and plaque compaction by microglia in 5xFAD mice.

SYK deletion in microglia leads to worsened neuronal health and memory impairment in 5xFAD mice

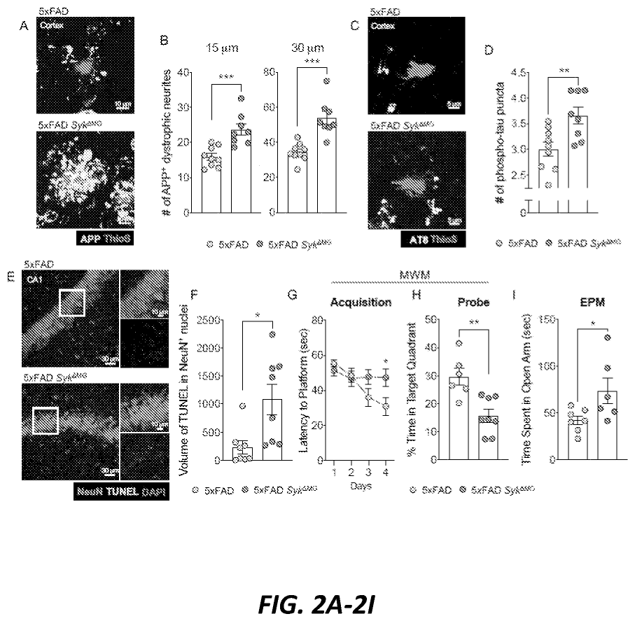

Due to the influence of SYK signaling in microglia on Aβ accumulation and composition, and the propensity of Aβ to impair neuronal function (Colie et al., 2017; Jawhar et al., 2012), neuronal health was assessed in 5xFAD and 5xFAD Syk^{ΔMG} mice. It was found that 5-month-old 5xFAD Syk^{ΔMG} mice had a ˜1.5-fold increase in plaque-associated dystrophic neurites in the cortex compared with 5xFAD controls (FIGS. 2A and 2B). Heightened accumulation of hyperphosphorylated tau, labeled with the AT8 antibody, was also observed around plaques in the cortex of 5xFAD Syk^{ΔMG} mice (FIGS. 2C and 2D). This increase in hyperphosphorylated tau is likely indicative of neuronal debilitation (Gendron and Petrucelli, 2009; Kanno et al., 2014) that has culminated from the accumulation of neurotoxic Aβ. 5xFAD Syk^{ΔMG} mouse neurons in the CA1 region of the hippocampus also displayed increased levels of cell death, as visualized by TUNEL staining (FIGS. 2E and 2F). These collective findings suggest that SYK activity in microglia helps to preserve neuronal health in 5xFAD mice.

To better understand how Syk-deficiency in microglia impacts brain function, the performance of 4-month-old 5xFAD and 5xFAD Syk^{ΔMG} mice was evaluated in the Morris water maze (MWM), which is commonly used to assess spatial learning and memory. In parallel with increased Aβ plaque load and neuronal death, it was found that 5xFAD Syk^{ΔMG} mice also displayed more severe cognitive impairments than 5xFAD littermate controls. Specifically, it was found that it took markedly longer for 5xFAD Syk^{ΔMG} mice to find the hidden platform in comparison to 5xFAD littermate controls on day 4 of the MWM test (FIG. 2G), which suggests that there is impaired spatial learning in 5xFAD Syk^{ΔMG} mice. These differences in performance were not due to altered bodyweight or defects in locomotor activity in 5xFAD Syk^{ΔMG} mice, as the body weight and travel velocities in the MWM were similar between the experimental mouse groups (Figure S20 and S2P). Moreover, when the platform was removed for a probe trial on day 5 of the MWM test, 5xFAD Syk^{ΔMG} mice spent significantly less time than 5xFAD mice in the quadrant of the pool where the hidden platform had previously been located (FIG. 2H), which is indicative of impaired spatial memory in 5xFAD Syk^{ΔMG} mice. These MWM findings suggest that disruption of microglial SYK signaling in 5xFAD mice leads to defective spatial learning and memory.

Aβ deposition in 5xFAD mice has also been shown to spur the development of risk-taking and exploratory behaviors, as can be observed in some AD patients (Ha et al., 2012; Jawhar et al., 2012). Therefore, to explore if targeted ablation of SYK in microglia also influences risk-taking and exploratory behaviors, the performance of 4-month-old 5xFAD Syk^{ΔMG} mice and 5xFAD littermate controls was evaluated in the elevated plus maze (EPM). In these studies, it was observed that 5xFAD Syk^{ΔMG} mice spent more time exploring the open arm of the maze compared to 5xFAD controls (FIGS. 2I and S2Q), which suggests that SYK deletion in microglia on the 5xFAD background also leads to greater levels of risk-taking and exploratory behaviors. Taken together, these data demonstrate a role for microglial SYK in preventing neuronal loss as well as limiting the development of memory impairment and risk-taking-related behaviors in Aβ-mediated neurodegenerative disease.

SYK regulates microglial proliferation and association with Aβ plaques

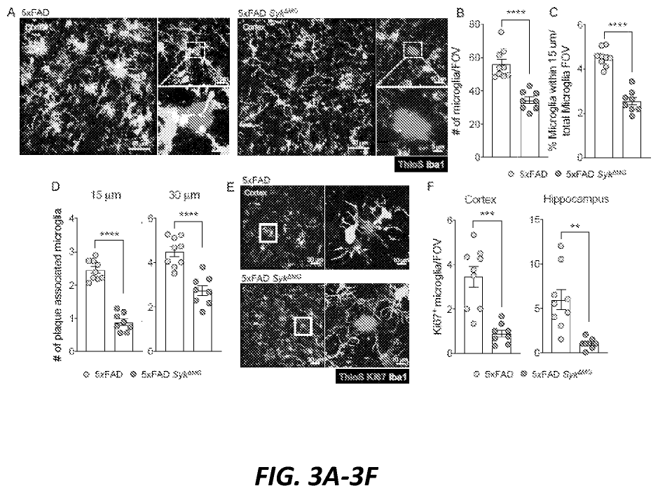

To gain insights into how SYK influences microglial biology in response to Aβ pathology, the impact of SYK deletion on microgliosis was explored. Here, it was found that 5xFAD littermate controls have significantly more cortical and hippocampal microglia than 5xFAD Syk^{ΔMG} mice (FIGS. 3A, 3B, S2C, and S2D). Impaired microglia clustering to Aβ plaques in the cortex and hippocampus of 5xFAD Syk^{ΔMG} mice was also observed, with the number of plaque-associated microglia being 2-fold lower in 5xFAD Syk^{ΔMG} mice than 5xFAD littermate controls (FIGS. 3A, 3C, 3D, S2C, and S2E). Interestingly, the reduction in microglia numbers observed in 5xFAD Syk^{ΔMG} mice appears to be specific to Aβ-mediated pathology, as no appreciable differences in microglia numbers between Syk^{ΔMG} mice and Cre-negative Cx3cr1^{ERT2Cre} Syk^{fl/fl} mice (Sykcon) which do not express the 5xFAD transgenes was observed (Figures S2R and S2S). In addition, 5xFAD mice that underwent delayed deletion of SYK at 4 months of age displayed a corresponding decrease in microglial number and association with Aβ plaques at 8 months of age compared with 5xFAD controls (Figures S2L-S2N). Thus, the SYK driving microglial responses exists during both disease-onset and disease-progression in 5xFAD mice. To distinguish what might contribute to the reduced numbers of microglia in 5xFAD Syk^{ΔMG} mice, the proliferative capacity of SYK-deficient microglia was evaluated. It was found that SYK deficiency in 5xFAD mice leads to reduced microglial proliferation, as illustrated by the ˜3-fold decrease in Ki67+ microglia seen in the cortex and hippocampus of 5xFAD Syk^{ΔMG} mice (FIGS. 3E and 3F). In contrast, in the absence of Aβ accumulation in mice that lack the 5xFAD transgenes, no appreciable differences were detected in microglial Ki67 staining between Sykcon and Syk^{ΔMG} mice (Figure S2R and S2T).

The possibility also exists that increased microglial death could be contributing at some level to reduced numbers of microglia seen in the brains of in 5xFAD Syk^{ΔMG} mice (FIGS. 3A and 3B). Therefore, to interrogate if deletion of SYK in microglia leads to increased levels of microglial cell death in the 5xFAD model, TUNEL staining was performed in the cortex of 5-month-old 5xFAD and 5xFAD Syk^{ΔMG} mice. No appreciable numbers of Iba1+ microglial cells that stained positive for TUNEL in either 5xFAD or 5xFAD Syk^{ΔMG} mice were detected (Figure S2W and S2X). This suggests that apoptosis is likely not a major driver of decreased microglial cell numbers in 5xFAD Syk^{ΔMG} mice at this time point. In summary, these results suggest that SYK is involved in coordinating microgliosis in response to Aβ pathology.

Aβ-induced microglial activation is impaired in the absence of SYK

To ascertain whether SYK signaling also impacts microglia activation in response to Aβ, differences in microglia morphology using a Sholl analysis were evaluated. Microglia morphology is widely used to offer insights into their activation status. For instance, homeostatic/resting microglia typically exhibit highly ramified processes, whereas activated microglia tend to retract these processes and acquire an ameboid morphology (Parakalan et al., 2012). Using a Sholl analysis to decipher morphological changes, it was observed that non-plaque-associated microglia in the cortex and hippocampus of 5xFAD Syk^{ΔMG} mice had significantly more ramified processes compared with 5xFAD controls (FIGS. 4A, 4B, S2F, and S2G). In contrast, resting morphological differences were not seen between Sykcon and Syk^{ΔMG} mice in the absence of Aβ (Figures S2U and S2V), which suggests that the morphological differences observed between 5xFAD Syk^{ΔMG} and 5xFAD controls is specific to Aβ-driven neurological disease. Taken together, these Sholl analysis results suggest that microglia intrinsic SYK signaling plays a central role in coordinating the ability of microglia to take on a morphologically activated state in response to Aβ pathology.

Recent studies have also shown that microglia upregulate a unique transcriptional program in neurodegenerative disease. This activation-induced transition into diseased-associated microglia (DAM) is thought to endow microglia with key neuroprotective functions (Keren-Shaul et al., 2017). This progressive shift from resting-state microglia to DAMs involves the coordinated downregulation of many homeostatic markers in stage 1, followed by an upregulation of genes related to microglial response to neurodegenerative pathology in stage 2. Therefore, it was next aimed to elucidate whether SYK affects DAM acquisition in response to Aβ-driven neuropathology. To answer this question and gain a comprehensive and unbiased picture of how SYK modulates microglial biology in 5xFAD mice, bulk RNA sequencing (RNA-seq) was performed on magnetic bead-sorted CD11b+ cells isolated from the brains of 5-month-old Sykcon, Syk^{ΔMG}, 5xFAD, and 5xFAD Syk^{ΔMG} mice (Figure S3A). These brains were cleared of their meninges and choroid plexus to eliminate contamination of potential SYK-deficient BAMs. Principal component (PC) analysis revealed, as expected, that 5xFAD microglia form a distinct cluster from control microglia (FIG. 4C), which is indicative of the altered transcriptional profile microglia take on in the presence of Aβ. In contrast, the loss of SYK in microglia blocked this transformation, as 5xFAD Syk^{ΔMG} microglia clustered with unperturbed Sykcon and Syk^{ΔMG} microglia, suggesting that 5xFAD Syk^{ΔMG} are more similar to homeostatic microglia than those isolated from the 5xFAD mouse model of AD (FIG. 4C). Upon further inspection, 2769 downregulated and 2668 upregulated genes (FDR<0.1) were observed when comparing microglia isolated from 5xFAD Syk^{ΔMG} and 5xFAD mice (FIGS. 4D and S3D). Moreover, KEGG analysis revealed that many of the repressed genes in 5xFAD Syk^{ΔMG} microglia were related to neurodegeneration (FIG. 4E). In contrast to the numerous transcriptional differences seen between 5xFAD and 5xFAD Syk^{ΔMG} microglia, only a marginal effect of SYK deletion was observed between Syk^{ΔMG} and Sykcon microglia, with 37 downregulated and 7 upregulated genes (FDR<0.1) (Figures S3B and S3C). These findings suggest that SYK acts as a regulator of the transcriptional shift that microglia undergo in response to Aβ-associated neuropathology in 5xFAD mice.

Notably, it was also found that a large number of genes were markedly repressed in 5xFAD Syk^{ΔMG} microglia (FIG. 4F). More specifically, upon Syk deletion a significant downregulation of stage 1 DAM genes between 5xFAD Syk^{ΔMG} and 5xFAD microglia was observed (FIG. 4F). However, an even more striking downregulation of stage 2 DAM genes (i.e., Lpl, Cst7, Itgax, Axl, Clec7a, Csfl, and Ccl6) was observed in 5xFAD Syk^{ΔMG} microglia relative to 5xFAD microglia (FIGS. 4F and S3D). Therefore, SYK appears to be needed for the ability of microglia to acquire the more activated stage 2 DAM transcriptional phenotype in 5xFAD mice. To validate this transcriptional block in DAM generation seen in SYK-deficient microglia at the protein level, immunofluorescence staining was performed to evaluate the expression levels of the signature microglial homeostatic marker, Tmem119. As DAMs undergo transcriptional activation, homeostatic Tmem119 expression canonically decreases in stage 1 DAMs (Krasemann et al., 2017). However, it was observed that 5xFAD Syk^{ΔMG} microglia retained significantly higher Tmem119 expression compared to 5xFAD microglia (Figures S3E and S3F), suggesting their retention of a homeostatic state. In addition, the expression of stage-2 DAM marker, CLEC7A (Krasemann et al., 2017), on Iba1+microglia surrounding Aβ plaques in 5xFAD and 5xFAD Syk^{ΔMG} mice was investigated. These imaging studies revealed significantly reduced expression of CLEC7A on Iba1+microglia surrounding plaques in 5xFAD Syk^{ΔMG} mice (Figures S3G-S31), suggesting that SYK is required for the optimal upregulation of this DAM protein. Altogether, these findings reveal that SYK centrally contributes to the transformation of homeostatic microglia into DAMs following exposure to Aβ.

The downstream signaling that coordinates DAM acquisition has remained poorly described, although, it has been suggested that PI3K/AKT signaling can regulate many of the processes and pathways linked to microglial activation in AD (Chu et al., 2021; Gabbouj et al., 2019; Hooper et al., 2008; Ulland et al., 2017). Therefore, how the loss of SYK in microglia regulates PI3K/AKT signaling in response to Aβ pathology was interrogated. Utilizing magnetic bead-sorted CD11b+ microglia isolated from the brains of 5-month-old 5xFAD and 5xFAD Syk^{ΔMG} mice that were stripped of their meninges and choroid plexus, the phosphorylation status of 18 proteins that have been shown to be centrally involved in the PI3K/AKT signaling pathway waws evaluated. In these studies, it was found that one particular arm of the AKT signaling pathway was differentially regulated between 5xFAD and 5xFAD Syk^{ΔMG} microglia. More specifically, it was observed that phosphorylation levels of both AKT (P-S473) and GSK3β (P-Ser9) were reduced in 5xFAD Syk^{ΔMG} microglia compared with 5xFAD control microglia (FIGS. 4G and 4H). These findings are notable as decreased phosphorylation of AKT (P-S473) and GSK3β (P-Ser9) have been observed in the brains of AD patients in comparison to age-matched controls (Mateo et al., 2006; Steen et al., 2005). Moreover, mutations in GSK3β have also been linked to both familial and sporadic forms of AD in humans (Schaffer et al., 2008). The data indicate that 5xFAD Syk^{ΔMG} microglia exhibit decreased AKT activation as well as diminished phosphorylation of GSK3ß at Ser9 (FIGS. 4G and 4H). Phosphorylation of GSK3ß at Ser9 leads to its potent inactivation (Doble and Woodgett, 2003; Steen et al., 2005), which ultimately indicates that there is increased activation of GSK3β in 5xFAD Syk^{ΔMG} microglia. Given that GSK3β activation has been shown to contribute to Aβ accumulation, tau phosphorylation, and neuronal damage in models of AD (DaRocha-Souto et al., 2012; Hernandez et al., 2013; Hurtado et al., 2012; Reddy, 2013), SYK-related modulation of the GSK3β pathway may contribute to the exacerbation of disease seen in 5xFAD Syk^{ΔMG} mice.

The extent to which SYK regulates microglial transcriptional transition into DAMs prompted an investigation into what upstream receptor SYK signaling relies on to enact Aβ pathology-induced microglial response. Extensive work has characterized microglial receptor TREM2 as influential in driving microglial acquisition of the DAM transcriptome (Keren-Shaul et al., 2017; Krasemann et al., 2017; Wang et al., 2015). Based on these findings, it was determined if the impaired transcriptional shift that was observed in the SYK-deficient 5xFAD microglia phenocopies the previously described deficiency in microglial transcriptional activation seen in TREM2-deficient 5xFAD microglia. To better understand the extent to which SYK regulates transcriptional DAM acquisition and to identify if TREM2 regulates SYK function, the RNA-seq dataset was compared with a previously published dataset analyzing 5xFAD Trem2−/− microglia (Griciuc et al., 2019). It was found that 25% of genes upregulated and ˜60% of genes downregulated in 5xFAD Trem2−/− microglia were shared with 5xFAD Syk^{ΔMG} microglia (FDR<0.05) (Figure S3J). In addition, the genes downregulated by 5xFAD Trem2^{-/-} and 5xFAD Syk^{ΔMG} microglia share molecular function terms such as “signaling receptor binding” and “protein binding” (Figure S3K). These data suggest a shared signaling axis between TREM2 and SYK. However, the transcriptional shift upon SYK deletion in 5xFAD Syk^{ΔMG} microglia encompasses a substantial population of uniquely upregulated (>97%) and downregulated (>63%) differentially expressed genes not observed in 5xFAD mice that lack TREM2 (FDR<0.05) (Figure S3J). Therefore, it is likely that TREM2 signaling through SYK is only partially regulating microglial DAM transition, and that SYK conceivably transmits signals from multiple receptors in addition to TREM2 in the 5xFAD mouse model.

Aβ pathology promotes increased lipid droplet formation and ROS production in SYK-deficient microglia

The RNA-seq findings revealed that 5xFAD Syk^{ΔMG} microglia exhibit a prominent reduction in lipoprotein lipase (Lpl) expression (FIG. 4F), a DAM-marker for regulating cellular lipid homeostasis and lipid droplet accumulation (Loving et al., 2021). Interestingly, recent work in microglial biology during aging and neurodegeneration has identified a population of lipid-droplet-accumulating microglia (LDAMs), which display impaired phagocytosis and increased reactive oxygen species (ROS) production (Marschallinger et al., 2020). Therefore, to further understand how SYK may regulate microglial responses in the AD-brain environment, lipid homeostasis was evaluated in SYK-deficient microglial using BODIPY which is a fluorescent dye that detects lipid droplets (Marschallinger et al., 2020). In these studies, a significant increase in BODIPY fluorescence was observed in 5xFAD Syk^{ΔMG} CD11b⁺CD45^{int} microglia, indicating an increase in lipid droplet accumulation in SYK-deficient microglia (Figures S4A and S4B). Previous work has defined several dysfunctions in LDAMs, including their increased production of reactive oxygen species (ROS) in the aged brain (Marschallinger et al., 2020). Indeed, 5xFAD Syk^{ΔMG} CD11b CD45^{int} microglia displayed an increase in CellROX fluorescence, a cell-permeant dye that fluoresces when oxidized by ROS, compared with control 5xFAD microglia (Figures S4C and S4D). These collective findings suggest that SYK may act to partially limit microglial transition to an LDAM-like state.

Phagocytosis of Aβ is coordinated by SYK

DAMs have been shown to highly express multiple genes that are involved in phagocytosis, and this upregulation of phagocytic machinery is thought to be one of the ways in which DAMs can exert neuroprotective clearance of neurotoxic material in degenerative disease (Keren-Shaul et al., 2017). Given that the loss of Syk limits microglia DAM marker expression and augments Aβ accumulation, it was hypothesized that SYK may also play roles in microglial phagocytosis of Aβ in 5xFAD mice, which could help to explain the elevated deposition of Aβ seen in 5xFAD Syk^{ΔMG} mice (FIG. 1). To investigate this possibility, Aβ engulfment by microglia was measured using immunohistochemistry and 5xFAD microglia were observed to engulf more than twice the amount of Aβ than 5xFAD Syk^{ΔMG} microglia (FIGS. 5A and 5B). Similarly, approximately twice the volume of Aβ was engulfed within CD68, a well-described marker of microglial phagolysosomes (Holness and Simmons, 1993; Walker and Lue, 2015; Zotova et al., 2011), in 5xFAD microglia compared with 5xFAD Syk^{ΔMG} microglia (FIGS. 5C and 5D). Notably, it was noticed that 5xFAD Syk^{ΔMG} and 5xFAD microglia upregulated CD68 to comparable total levels across the cortex (Figures S4E and S4F). However, while much of the engulfed Aβ detected in 5xFAD microglia colocalized with CD68, there was far less Aβ engulfed within CD68 in SYK-deficient 5xFAD microglia (FIGS. 5C and 5D).

These imaging data indicate that SYK is a regulator of Aβ phagocytosis in microglia. To further substantiate a role for microglial SYK in Aβ phagocytosis, this was explored in a secondary experimental system. In these studies, 5-month-old 5xFAD Syk^{ΔMG} mice and 5xFAD littermate controls received an intraperitoneal (i.p.) injection of Methoxy-X04 which is a brain penetrant dye that labels fibrillar Aβ. After 3 hours, brains were harvested from these mice to quantify the percentage of microglia that had taken up Methoxy-X04-labelled Aβ using flow cytometry. Approximately 20% of 5xFAD microglia had ingested Aβ (Methoxy-X04*) while almost none of the 5xFAD Syk^{ΔMG} microglia contained Methoxy-X04-stained Aβ (FIGS. 5E and 5F). In total, these combined results provide evidence that SYK promotes the phagocytic capacity of microglia in response to Aβ.

What contributes to defective Aβ phagocytosis in 5xFAD Syk mice was next explored. GSK3β signaling was investigated as this was found to be profoundly affected in 5xFAD Syk^{ΔMG} microglia (FIGS. 4G and 4H). To this end, WT and SYK-deficient bone marrow-derived macrophages (BMDMS) were pre-treated with the GSK3ß inhibitor Tideglusib and then evaluated phagocytosis of CypHer5E-tagged Aβ oligomers. CypHer5E fluoresces in a low pH environment such as the phagolysosome; therefore, CypHer5E fluorescence was analyzed by flow cytometry as a readout for Aβ phagocytosis. In these studies, it was found that GSK3β inhibition with Tideglusib treatment significantly increased Aβ phagocytosis in SYK-deficient macrophages (FIGS. 5G and 5H). This suggests that the dysregulated GSK3β signaling that unfolds in the absence of SYK can contribute to defective phagocytosis of Aβ by macrophages.

Exogenous activation of the CLEC7A-SYK signaling pathway promotes improved clearance of Aβ

Thus far, it has been demonstrated that SYK-deficiency impairs microglial responses to Aβ pathology in 5xFAD mice. However, to reinforce the integral role for SYK in driving microglial response in this environment, it was investigated wither the reciprocal activation of SYK signaling would enhance protective microglial activities in the AD brain. To achieve this, SYK activation was induced through CLEC7A, a receptor shown to be important for microglial activation in response to AD pathology. CLEC7A is a canonical fungal pathogen receptor that signals through SYK in the periphery and has recently been identified to be upregulated in Stage 2 DAMs (Drummond et al., 2011; Keren-Shaul et al., 2017). In the studies presented here, SYK was identified as playing a role in Aβ phagocytosis (FIGS. 5A-H); therefore, it was investigated whether CLEC7A-stimulated SYK signaling could enhance microglial phagocytic response. Thus, pustulan, a β-D-glucan and ligand for CLEC7A, was injected into the hippocampus of 2-month-old 5xFAD and 5xFAD Syk^{ΔMG}ì mice. The hippocampus was chosen due to its reliable accumulation of Aβ in 5xFAD mice. As an internal control, one hemisphere of the hippocampus received a vehicle injection, while the other hippocampal hemisphere received a pustulan injection. After 7 days, the brains were harvested from the injected mice and levels of Aβ was investigated between the vehicle and pustulan-injected hippocampal hemispheres using immunofluorescence (FIG. 5I). Strikingly, 5xFAD mice displayed decreased Aβ load in the pustulan-injected hippocampal hemisphere compared to the vehicle-injected hippocampal hemisphere (FIGS. 5J and 5K), indicating that pustulan-induced microglial CLEC7A activation is sufficient in stimulating Aβ clearance in the 5xFAD brain. In contrast, pustulan treatment in 5xFAD Syk^{ΔMG} mice did not promote Aβ clearance in the hippocampus (FIGS. 5J and 5L), suggesting that SYK is necessary for the protective CLEC7A-driven phagocytic response by microglia. Altogether, the data suggests that CLEC7A signals through SYK to promote protective microglial phagocytic response to Aβ.

DAM generation is regulated by SYK in demyelinating neuroinflammatory disease

Next, it was investigated whether SYK also influences DAM generation and microglial biology in other models of neurological disease. As a first approach, the impact of SYK deletion was explored in microglia on demyelinating neuroinflammatory disease progression in the experimental autoimmune encephalomyelitis (EAE) mouse model of multiple sclerosis (MS). Microglia have previously been described to play pivotal roles in EAE disease progression (Plastini et al., 2020). In particular, the clearance of myelin debris by microglia is believed to be needed to limit neuronal damage in EAE (Cignarella et al., 2020; Takahashi et al., 2007; Weinger et al., 2011). In the studies, it was found that Syk^{ΔMG} mice develop exacerbated paralyzing disease and more severe demyelination in comparison to Sykcon littermate controls (FIGS. 6A-C). Interestingly, ablation of SYK in Syk^{ΔMG} mice was also found to have an effect on T cell responses in the EAE disease model. More specifically, a modest, albeit statistically significant, increase in total T cell numbers, and more T cells producing GM-CSF, IFN-γ, and IL-17 in the spinal cords of Syk^{ΔMG} EAE mice relative to Sykcon EAE controls during the effector phase of EAE disease (>30 days post-immunization) was observed (Figure S5A-S5E). Moreover, less splenic CD4⁺ T cells making GM-CSF, IFNγ, and IL-17 in Syk^{ΔMG} EAE mice compared to Sykcon EAE controls when mice were harvested during the EAE effector phase was detected (Figure S5F-S5H). These collective findings point toward key neuroprotective roles for SYK in microglia during demyelinating neuroinflammatory disease.

Having seen that SYK ablation in Syk^{ΔMG} mice leads to more severe demyelinating neuroinflammatory disease, it was sought to better understand how SYK influences microglial responses in EAE. Given the results in the 5xFAD model, was interesting to interrogate whether SYK also instructs DAM generation and modulates microglial transcriptional expression of phagocytic machinery in this separate model of neurodegenerative disease. To accomplish this in a comprehensive and unbiased fashion, single-cell RNA-sequencing (scRNA-seq) was conducted on sorted spinal cord macrophages. In comparison to the 5xFAD AD mouse model, where it has been shown that there is negligible recruitment of peripherally derived myeloid cells into the CNS (Reed-Geaghan et al., 2020; Wang et al., 2016), disease progression in EAE is characterized by massive infiltration of various circulating myeloid cell types into the spinal cord (Constantinescu et al., 2011). This ultimately makes it challenging to unequivocally differentiate between bona fide microglia and CNS-infiltrating myeloid cells using traditional cell sorting techniques (e.g., flow cytometric sorting on CD11b+ cells). Therefore, to circumvent this technical limitation associated with the EAE model, Syk^{+/+} Cx3cr1^{ERT2Cre} and Syk^{fl/fl} Cx3cr1^{ERT2Cre} mice were crossed with the Ai6-ZsGreen reporter mouse line to target Ai6-Zsgreen expression to Cx3cr1-expressing cells. The Ai6-ZsGreen model has been adopted by others in the field to purify CNS-resident macrophages in settings where peripheral-derived myeloid cells are expected to infiltrate the CNS (Batista et al., 2020; Whittaker Hawkins et al., 2017). Ai6-ZsGreen reporter mice possess a LoxP-flanked STOP cassette that prevents the expression of green fluorescent protein variant ZsGreen1 until the stop site is excised after tamoxifen induction of Cre-recombinase activity. Following the withdrawal of tamoxifen, short-lived Ai6-ZsGreen expressing cells in the periphery are promptly replaced by newly derived cells that lack Ai6-ZsGreen expression. In contrast, the long-lived nature of microglia allows them to retain their Ai6-ZsGreen signal for months post-tamoxifen withdrawal (Goldmann et al., 2013).

In the studies, we purified ZsGreen+ cells from the spinal cords of Ai6-ZsGreen Syk^{+/+} Cx3cr1^{ERT2Cre} (Sykcon-Ai6) and Ai6-ZsGreen Syk^{fl/fl} Cx3cr1^{ERT2Cre} (Syk^{ΔMG-Ai6}) mice at day post-EAE induction using flow cytometry-based cell sorting (Figure S5I). Utilizing scRNA-seq, 6 unique microglia populations were uncovered, including homeostatic microglia, highly metabolic microglia, MI-like microglia, and M2-like microglia (FIG. 6D). A unique population of microglia was also identified and named CD36hi microglia, as Cd36 was one of the top-defining genes of this cluster and it failed to conform with other known microglia types (FIGS. 6D and 6E). The final microglia cluster highly expressed the canonical genes of DAMs, including Cst7, Lpl, Spp1, Tyrobp, and Itgax (FIGS. 6D and 6E).

To understand if microglia followed a trajectory in their maturation state during EAE and if this was influenced by SYK, a pseudotime analysis of the microglia transcriptional data was performed. After establishing homeostatic microglia as the earliest point in pseudotime, three potential pathways were revealed: a homeostatic to highly metabolic and M1-like microglia pathway, a homeostatic to CD36hi pathway, and a homeostatic to DAM and M2-like pathway (FIG. 6F). When comparing the distribution of cells in each cluster by Syk^{con-Ai6} vs. Syk^{ΔMG-Ai6}, it was observed that Sykcon-Ai6 microglia tend to follow the homeostatic to DAM and M2-like pathways more than Syk^{ΔMG-Ai6} microglia (FIGS. 6F and 6G). In contrast, Syk^{ΔMG-Ai6} microglia, when compared to Syk^{con-Ai6} microglia, more commonly followed the homeostatic to CD36hi trajectory (FIGS. 6F and 6G). These pathway biases are confirmed by the proportion of cells in each cluster by sample, where the Syk^{con-Ai6} samples have a higher proportion of DAM microglia and the Syk^{ΔMG-Ai6} samples have a higher proportion of CD36hi microglia and few DAMs (FIG. 6H). Therefore, Syk^{ΔMG-Ai6} microglia appear to be less apt to acquire DAM transcriptional status in the EAE model of demyelinating neuroinflammatory disease.

Interestingly, the Syk^{ΔMG-Ai6} samples also have a higher proportion of homeostatic microglia when compared to microglia isolated from Syk^{con-Ai6} mice (FIG. 6H). To further examine the failure of Syk^{ΔMG-Ai6} microglia to take on a DAM transcriptional signature, feature plots were generated to visualize gene expression of DAM markers: (Cst7, Lpl, Itgax, and Spp1 by cluster (FIG. 6I). It was observed that Syk^{ΔMG} microglia in the DAM cluster had much lower average expression of these DAM genes compared with microglia obtained from Syk^{con-Ai6} mice (FIG. 6I). Finally, to better understand the biological processes potentially being driven by the DAM cluster, DAM-defining genes were used to plot KEGG and GO terms related to phagocytosis. These shared terms included “phagosome,” “abnormal phagocyte morphology,” and “microglia pathogen phagocytosis pathway,” and shared phagocytosis genes such as Trem2, Tyrobp, C1qa, Lamp1, Cd68, and Cd22 (FIG. 6J). In summary, the collective EAE findings corroborate the 5xFAD data characterizing SYK as a pivotal intracellular regulator of DAM generation and promoter of neuroprotective functions in microglia during neurodegenerative disease.

Defective SYK signaling in microglia during demyelinating disease causes damaged myelin debris accumulation and impaired oligodendrocyte proliferation

To further validate the ability of SYK to modulate microglial responses in a separate model of demyelinating disease that does not involve autoimmune attack, the effects of cuprizone intoxication was explored on neurological disease in Syk^{ΔMG} mice and Syk^{con} littermate controls. Cuprizone is toxic to myelinating oligodendrocytes, including those found in the corpus callosum, thus 5 continuous weeks of feeding cuprizone to mice leads to a localized areas of demyelination (Zhan et al., 2020). No appreciable changes were observed in myelin basic protein (MBP) levels in the corpus callosum of Syk^{ΔMG} and Syk^{con} mice, indicating that myelination at steady-state is not affected in SYK-deficient mice (Figures S6A and S6B). However, it was noticed that the corpus callosum of Syk^{ΔMG} mice had significantly fewer microglia than Syk^{con} controls during both cuprizone-induced demyelination and remyelination (Figures S6C and S6D). It was determined that the decreased number of microglia in Syk^{ΔMG} mice is likely not due to differential apoptosis using TUNEL staining (Figures S6E and S6F). Consistent with previous studies, it was found that feeding wild-type Sykcon mice cuprizone diet leads to increased staining of the phagocytic marker CD68 in Iba1+ cells (Figures S6G and S6H) (Cignarella et al., 2020). In contrast, CD68 positivity was substantially decreased in Iba1+ cells found in the corpus callosum of cuprizone-fed Syk^{ΔMG}ì mice during the demyelinating phase (Figures S6G and S6H), suggesting impaired phagocytic microglial response compared to Syk^{con} mice. Indeed, increased damaged myelin basic protein (dMBP) accumulation was evident during both demyelination and remyelination in the Syk^{ΔMG} corpus callosum compared with Syk^{con} mice (FIGS. 7A and 7B). Microglia are established to phagocytose damaged myelin in the cuprizone model of demyelination; therefore, this accumulation is likely due to a microglial phagocytic deficit (Gudi et al., 2014). These results provide evidence that SYK signaling in microglia is involved in the clearance of myelin debris independent of the robust autoimmune response associated with EAE.

The inability to phagocytose myelin debris in the cuprizone model is known to obstruct aspects of oligodendrocyte biology including the differentiation of oligodendrocyte precursor cells (OPCs) into myelin-producing oligodendrocytes (Back et al., 2005). Therefore, it was hypothesized that any phagocytic deficits seen in Syk^{ΔMG} mice in the cuprizone model would subsequently manifest as impaired OPC proliferation and/or differentiation into mature oligodendrocytes during the remyelination phase that follows cuprizone cessation. Consistent with this hypothesis, it was found that SYK deficiency in cuprizone-treated Syk^{ΔMG} mice leads to greatly reduced numbers of OPCs (Olig2⁺Pdgfra⁺ cells) and oligodendrocytes (Olig2⁺CC1⁺ cells) during the remyelination phase of the cuprizone model (FIGS. 7C-7E). It was also noted that OPCs in cuprizone-treated Syk^{ΔMG} mice had severely impaired proliferation during demyelination (Figures S6I and S6J), which likely accounts for the decreased numbers of OPCs and oligodendrocytes seen in Syk^{ΔMG} mice following cuprizone treatment. In comparison, comparable numbers of OPCs and oligodendrocytes in Syk^{ΔMG} mice and Syk^{con} littermate controls that were fed normal chow was observed (FIGS. 7C-E), suggesting that SYK deficiency in microglia does not appreciably affect oligodendrocyte-lineage cell numbers under homeostatic conditions. The findings indicate that disruption of SYK signaling in microglia causes prominent defects in the clearance of damaged myelin in the cuprizone model of demyelinating disease. Moreover, they suggest that the lack of neuroprotective functions in SYK-deficient microglia can ultimately lead to impaired oligodendrocyte generation during remyelination. Altogether, these cuprizone data support the 5xFAD and EAE findings that define a role for SYK in promoting protective microglial responses that limit neurodegenerative disease progression.

### Discussion

Human GWAS and sequencing studies conducted over the last few years have helped to uncover an ever-increasing link between microglial dysfunction and various neurodegenerative disorders (Guerreiro et al., 2013; Jonsson et al., 2013; Keren-Shaul et al., 2017; Podlesny-Drabiniok et al., 2020). This has spurred tremendous recent efforts to reveal how microglial biology contributes to neurodegenerative disease. For instance, the identification of the link between mutations in TREM2 and (D) 33 in human AD has led to breakthroughs in the understanding of how these microglial receptors affect neurobiology in multiple neurodegenerative disease models (Bradshaw et al., 2013; Takahashi et al., 2007; Wang et al., 2015; Wang et al., 2016). While great strides have been made in recent years in defining some of the surface receptors that modulate microglial biology in neurodegenerative disease, the identity of the key intracellular signaling molecules exploited by microglia to regulate their neuroprotective functions is currently less well understood. In the studies presented here, SYK has been identified as an intracellular regulator of neuroprotective microglial responses in mouse models of both AD and MS.

SYK is perhaps best known for its roles in the generation of protective immunity against many fungal infections as well as in the regulation of T cell and B cell receptor signaling (Cornall et al., 2000; Latour et al., 1997; Malik et al., 2018). However, in recent years there has been growing appreciation for the involvement of SYK in models of sterile inflammation that do not involve microbes (Chung et al., 2019). In particular, it has been shown that SYK activation occurs following stimulation with various damage-associated molecular patterns (DAMPs) released from damaged or dying cells and that SYK-directed signaling in these scenarios induces robust immune responses (Mocsai et al., 2010; Serbulea et al., 2018). While there has been some initial progress made in identifying key roles for SYK in sterile inflammatory disorders that affect peripheral organs, far less is known in regard to how SYK signaling affects neurological health and disease. That being said, the SYK homolog in Drosophila, known as Shark, was shown to play a role in glial phagocytosis of axonal debris (Ziegenfuss et al., 2008). In addition, there have been a handful of recent studies that have used pharmacological inhibitors and in vitro cell culture systems to begin to explore how SYK affects CNS biology (Paris et al., 2014). For instance, it has been demonstrated that SYK activation, as indicated by phospho-SYK staining, is highly upregulated in plaque- and tau-associated microglia (Schweig et al., 2017).

The role of SYK in demyelinating neuroinflammatory disease also currently remains poorly described. This is surprising given that mutations in various SYK-related molecules have been identified as prominent MS genetic risk factors (International Multiple Sclerosis Genetics et al., 2011; Ramagopalan et al., 2010). For instance, mutations in multiple upstream activators of SYK including CD37, TREM2, numerous C-type lectin receptors (e.g., CLEC16A, CLECL1, and CLEC2D), and Fc receptor-like proteins (i.e., FCRL2 and FCRL3) have been strongly linked to MS in GWAS studies (International Multiple Sclerosis Genetics, 2019; International Multiple Sclerosis Genetics et al., 2011). Likewise, mutations in downstream molecules involved in SYK signaling including BCL10 and MALTI have also been associated with MS risk (Mc Guire et al., 2013; Molinero et al., 2012).

In summary, while pivotal roles for microglia have recently been uncovered in AD, MS, and many other neurodegenerative disorders (Karch and Goate, 2015; Krasemann et al., 2017; Malik et al., 2013; Pluvinage et al., 2019; Wang et al., 2015; Zhong et al., 2019), the key signaling pathways that microglia leverage to instruct neuroprotective functions remain poorly defined. Through the studies presented here, SYK has been identified as an instrumental regulator of neuroprotective responses in AD and MS. Moreover, the studies demonstrate that targeting SYK may offer novel strategies to boost microglial protective responses, including phagocytosis of neurotoxic material, to treat neurodegenerative disease.

### BIBLIOGRAPHY FOR EXAMPLE I

Abram, C. L., and Lowell, C. A. (2007). The expanding role for ITAM-based signaling pathways in immune cells. Sci STKE 2007, re2.
Back, S. A., Tuohy, T. M., Chen, H., Wallingford, N., Craig, A., Struve, J., Luo, N. L., Banine, F., Liu, Y., Chang, A., et al. (2005). Hyaluronan accumulates in demyelinated lesions and inhibits oligodendrocyte progenitor maturation. Nat Med 11, 966-972.
Batista, S. J., Still, K. M., Johanson, D., Thompson, J. A., O'Brien, C. A., Lukens, J. R., and Harris, T. H. (2020). Gasdermin-D-dependent IL-1 alpha release from microglia promotes protective immunity during chronic Toxoplasma gondii infection. Nat Commun 11, 3687.
Bemiller, S. M., McCray, T. J., Allan, K., Formica, S. V., Xu, G., Wilson, G., Kokiko-Cochran, O. N., Crish, S. D., Lasagna-Reeves, C. A., Ransohoff, R. M., et al. (2017). TREM2 deficiency exacerbates tau pathology through dysregulated kinase signaling in a mouse model of tauopathy. Mol Neurodegener 12, 74.
Bradshaw, E. M., Chibnik, L. B., Keenan, B. T., Ottoboni, L., Raj, T., Tang, A., Rosenkrantz, L. L., Imboywa, S., Lee, M., Von Korff, A., et al. (2013). CD33 Alzheimer's disease locus: altered monocyte function and amyloid biology. Nat Neurosci 16, 848-850.
Brown, M. R., Radford, S. E., and Hewitt, E. W. (2020). Modulation of beta-Amyloid Fibril Formation in Alzheimer's Disease by Microglia and Infection. Front Mol Neurosci 13, 609073.
Chen, S. H., Tian, D. Y., Shen, Y. Y., Cheng, Y., Fan, D. Y., Sun, H. L., He, C. Y., Sun, P. Y., Bu, X. L., Zeng, F., et al. (2020). Amyloid-beta uptake by blood monocytes is reduced with ageing and Alzheimer's disease. Transl Psychiatry 10, 423.
Chu, E., Mychasiuk, R., Hibbs, M. L., and Semple, B. D. (2021). Dysregulated phosphoinositide 3-kinase signaling in microglia: shaping chronic neuroinflammation. J Neuroinflammation 18, 276.
Chung, C. G., Lee, H., and Lee, S. B. (2018). Mechanisms of protein toxicity in neurodegenerative diseases. Cell Mol Life Sci 75, 3159-3180.
Chung, Y. H., Kim, H. Y., Yoon, B. R., Kang, Y. J., and Lee, W. W. (2019). Suppression of Syk activation by resveratrol inhibits MSU crystal-induced inflammation in human monocytes. J Mol Med (Berl) 97, 369-383.
Cignarella, F., Filipello, F., Bollman, B., Cantoni, C., Locca, A., Mikesell, R., Manis, M., Ibrahim, A., Deng, L., Benitez, B. A., et al. (2020). TREM2 activation on microglia promotes myelin debris clearance and remyelination in a model of multiple sclerosis. Acta Neuropathol 140, 513-534.
Clark, E. A., and Giltiay, N. V. (2018). CD22: A Regulator of Innate and Adaptive B Cell Responses and Autoimmunity. Front Immunol 9, 2235.
Colie, S., Sarroca, S., Palenzuela, R., Garcia, I., Matheu, A., Corpas, R., Dotti, C. G., Esteban, J. A., Sanfeliu, C., and Nebreda, A. R. (2017). Neuronal p38alpha mediates synaptic and cognitive dysfunction in an Alzheimer's mouse model by controlling beta-amyloid production. Sci Rep 7, 45306.
Condello, C., Yuan, P., and Grutzendler, J. (2018). Microglia-Mediated Neuroprotection, TREM2, and Alzheimer's Disease: Evidence From Optical Imaging. Biol Psychiatry 83, 377-387.
Condello, C., Yuan, P., Schain, A., and Grutzendler, J. (2015). Microglia constitute a barrier that prevents neurotoxic protofibrillar Abeta42 hotspots around plaques. Nat Commun 6, 6176.
Constantinescu, C. S., Farooqi, N., O'Brien, K., and Gran, B. (2011). Experimental autoimmune encephalomyelitis (EAE) as a model for multiple sclerosis (MS). Br J Pharmacol 164, 1079-1106.
Cooper-Knock, J., Green, C., Altschuler, G., Wei, W., Bury, J. J., Heath, P. R., Wyles, M., Gelsthorpe, C., Highley, J. R., Lorente-Pons, A., et al. (2017). A data-driven approach links microglia to pathology and prognosis in amyotrophic lateral sclerosis. Acta Neuropathol Commun 5, 23.
Cornall, R. J., Cheng, A. M., Pawson, T., and Goodnow, C. C. (2000). Role of Syk in B-cell development and antigen-receptor signaling. Proc Natl Acad Sci USA 97, 1713-1718.
Da Mesquita, S., Louveau, A., Vaccari, A., Smirnov, I., Cornelison, R. C., Kingsmore, K. M., Contarino, C., Onengut-Gumuscu, S., Farber, E., Raper, D., et al. (2018). Functional aspects of meningeal lymphatics in ageing and Alzheimer's disease. Nature 560, 185-191.
DaRocha-Souto, B., Coma, M., Perez-Nievas, B. G., Scotton, T. C., Siao, M., Sanchez-Ferrer, P., Hashimoto, T., Fan, Z., Hudry, E., Barroeta, I., et al. (2012). Activation of glycogen synthase kinase-3 beta mediates beta-amyloid induced neuritic damage in Alzheimer's disease. Neurobiol Dis 45, 425-437.
Deczkowska, A., Keren-Shaul, H., Weiner, A., Colonna, M., Schwartz, M., and Amit, I. (2018). Disease-Associated Microglia: A Universal Immune Sensor of Neurodegeneration. Cell 173, 1073-1081.
Doble, B. W., and Woodgett, J. R. (2003). GSK-3: tricks of the trade for a multi-tasking kinase. J Cell Sci 116, 1175-1186.
Drummond, R. A., Saijo, S., Iwakura, Y., and Brown, G. D. (2011). The role of Syk/CARD9 coupled C-type lectins in antifungal immunity. Eur J Immunol 41, 276-281.
Efthymiou, A. G., and Goate, A. M. (2017). Late onset Alzheimer's disease genetics implicates microglial pathways in disease risk. Mol Neurodegener 12, 43.
Estus, S., Shaw, B. C., Devanney, N., Katsumata, Y., Press, E. E., and Fardo, D. W. (2019). Evaluation of CD33 as a genetic risk factor for Alzheimer's disease. Acta Neuropathol 138, 187-199.
Ferreira, S. T., Lourenco, M. V., Oliveira, M. M., and De Felice, F. G. (2015). Soluble amyloid-beta oligomers as synaptotoxins leading to cognitive impairment in Alzheimer's disease. Front Cell Neurosci 9, 191.
Gabbouj, S., Ryhanen, S., Marttinen, M., Wittrahm, R., Takalo, M., Kemppainen, S., Martiskainen, H., Tanila, H., Haapasalo, A., Hiltunen, M., et al. (2019). Altered Insulin Signaling in Alzheimer's Disease Brain-Special Emphasis on PI3K-Akt Pathway. Front Neurosci 13, 629.
Gate, D., Saligrama, N., Leventhal, O., Yang, A. C., Unger, M. S., Middeldorp, J., Chen, K., Lehallier, B., Channappa, D., De Los Santos, M. B., et al. (2020). Clonally expanded CD8 T cells patrol the cerebrospinal fluid in Alzheimer's disease. Nature 577, 399-404.
Gendron, T. F., and Petrucelli, L. (2009). The role of tau in neurodegeneration. Mol Neurodegener 4, 13.
Goldmann, T., Wieghofer, P., Muller, P. F., Wolf, Y., Varol, D., Yona, S., Brendecke, S. M., Kierdorf, K., Staszewski, O., Datta, M., et al. (2013). A new type of microglia gene targeting shows TAKI to be pivotal in CNS autoimmune inflammation. Nat Neurosci 16, 1618-1626.
Griciuc, A., Patel, S., Federico, A. N., Choi, S. H., Innes, B. J., Oram, M. K., Cereghetti, G., McGinty, D., Anselmo, A., Sadreyev, R. I., et al. (2019). TREM2 Acts Downstream of CD33 in Modulating Microglial Pathology in Alzheimer's Disease. Neuron 103, 820-835 e827.
Gu, L., and Guo, Z. (2013). Alzheimer's Abeta42 and Abeta40 peptides form interlaced amyloid fibrils. J Neurochem 126, 305-311.
Gudi, V., Gingele, S., Skripuletz, T., and Stangel, M. (2014). Glial response during cuprizone-induced de- and remyelination in the CNS: lessons learned. Front Cell Neurosci 8, 73.
Guerreiro, R., Wojtas, A., Bras, J., Carrasquillo, M., Rogaeva, E., Majounie, E., Cruchaga, C., Sassi, C., Kauwe, J. S., Younkin, S., et al. (2013). TREM2 variants in Alzheimer's disease. N Engl J Med 368, 117-127.
Ha, J., Kim, E. J., Lim, S., Shin, D. W., Kang, Y. J., Bae, S. M., Yoon, H. K., and Oh, K. S. (2012). Altered risk-aversion and risk-taking behaviour in patients with Alzheimer's disease. Psychogeriatrics 12, 151-158.
Hadas, S., Spira, M., Hanisch, U. K., Reichert, F., and Rotshenker, S. (2012). Complement receptor-3 negatively regulates the phagocytosis of degenerated myelin through tyrosine kinase Syk and cofilin. J Neuroinflammation 9, 166.
Hernandez, F., Lucas, J. J., and Avila, J. (2013). GSK3 and tau: two convergence points in Alzheimer's disease. J Alzheimers Dis 33 Suppl 1, S141-144.
Hickman, S., Izzy, S., Sen, P., Morsett, L., and El Khoury, J. (2018). Microglia in neurodegeneration. Nat Neurosci 21, 1359-1369.
Hickman, S. E., Allison, E. K., Coleman, U., Kingery-Gallagher, N. D., and El Khoury, J. (2019). Heterozygous CX3CR1 Deficiency in Microglia Restores Neuronal beta-Amyloid Clearance Pathways and Slows Progression of Alzheimer's Like-Disease in PS1-APP Mice. Front Immunol 10, 2780.
Holness, C. L., and Simmons, D. L. (1993). Molecular cloning of CD68, a human macrophage marker related to lysosomal glycoproteins. Blood 81, 1607-1613.
Hooper, C., Killick, R., and Lovestone, S. (2008). The GSK3 hypothesis of Alzheimer's disease. J Neurochem 104, 1433-1439.
Huang, Y., Happonen, K. E., Burrola, P. G., O'Connor, C., Hah, N., Huang, L., Nimmerjahn, A., and Lemke, G. (2021). Microglia use TAM receptors to detect and engulf amyloid beta plaques. Nat Immunol 22, 586-594.
Hurtado, D. E., Molina-Porcel, L., Carroll, J. C., Macdonald, C., Aboagye, A. K., Trojanowski, J. Q., and Lee, V. M. (2012). Selectively silencing GSK-3 isoforms reduces plaques and tangles in mouse models of Alzheimer's disease. J Neurosci 32, 7392-7402.
International Multiple Sclerosis Genetics, C. (2019). Multiple sclerosis genomic map implicates peripheral immune cells and microglia in susceptibility. Science 365.
International Multiple Sclerosis Genetics, C., Wellcome Trust Case Control, C., Sawcer, S., Hellenthal, G., Pirinen, M., Spencer, C. C., Patsopoulos, N. A., Moutsianas, L., Dilthey, A., Su, Z., et al. (2011). Genetic risk and a primary role for cell-mediated immune mechanisms in multiple sclerosis. Nature 476, 214-219.
Jawhar, S., Trawicka, A., Jenneckens, C., Bayer, T. A., and Wirths, O. (2012). Motor deficits, neuron loss, and reduced anxiety coinciding with axonal degeneration and intraneuronal Abeta aggregation in the 5XFAD mouse model of Alzheimer's disease. Neurobiol Aging 33, 196 e129-140.
Jonsson, T., Stefansson, H., Steinberg, S., Jonsdottir, I., Jonsson, P. V., Snaedal, J., Bjornsson, S., Huttenlocher, J., Levey, A. I., Lah, J. J., et al. (2013). Variant of TREM2 associated with the risk of Alzheimer's disease. N Engl J Med 368, 107-116.
Kanno, T., Tsuchiya, A., and Nishizaki, T. (2014). Hyperphosphorylation of Tau at Ser396 occurs in the much earlier stage than appearance of learning and memory disorders in 5XFAD mice. Behav Brain Res 274, 302-306.
Karch, C. M., and Goate, A. M. (2015). Alzheimer's disease risk genes and mechanisms of disease pathogenesis. Biol Psychiatry 77, 43-51.
Keren-Shaul, H., Spinrad, A., Weiner, A., Matcovitch-Natan, O., Dvir-Szternfeld, R., Ulland, T. K., David, E., Baruch, K., Lara-Astaiso, D., Toth, B., et al. (2017). A Unique Microglia Type Associated with Restricting Development of Alzheimer's Disease. Cell 169, 1276-1290 e1217.
Kimura, Y., Chihara, K., Honjoh, C., Takeuchi, K., Yamauchi, S., Yoshiki, H., Fujieda, S., and Sada, K. (2014). Dectin-1-mediated signaling leads to characteristic gene expressions and cytokine secretion via spleen tyrosine kinase (Syk) in rat mast cells. J Biol Chem 289, 31565-31575.
Klein, A. M., Kowall, N. W., and Ferrante, R. J. (1999). Neurotoxicity and oxidative damage of beta amyloid 1-42 versus beta amyloid 1-40 in the mouse cerebral cortex. Ann N Y Acad Sci 893, 314-320.
Konishi, H., and Kiyama, H. (2018). Microglial TREM2/DAP12 Signaling: A Double-Edged Sword in Neural Diseases. Front Cell Neurosci 12, 206.
Krasemann, S., Madore, C., Cialic, R., Baufeld, C., Calcagno, N., El Fatimy, R., Beckers, L., O'Loughlin, E., Xu, Y., Fanek, Z., et al. (2017). The TREM2-APOE Pathway Drives the Transcriptional Phenotype of Dysfunctional Microglia in Neurodegenerative Diseases. Immunity 47, 566-581 e569.
Lammert C R, Frost E L, Bellinger C E, Bolte A C, McKee C A, Hurt M E, Paysour M J, Ennerfelt H E, and Lukens J R (2020). AIM2 inflammasome surveillance of DNA damage shapes neurodevelopment. Nature 580, 647-652. 10.1038/s41586-020-2174-3. [PubMed: 32350463]
Lampron, A., Larochelle, A., Laflamme, N., Prefontaine, P., Plante, M. M., Sanchez, M. G., Yong, V. W., Stys, P. K., Tremblay, M. E., and Rivest, S. (2015). Inefficient clearance of myelin debris by microglia impairs remyelinating processes. J Exp Med 212, 481-495.
Latour, S., Fournel, M., and Veillette, A. (1997). Regulation of T-cell antigen receptor signalling by Syk tyrosine protein kinase. Mol Cell Biol 17, 4434-4441.
Lawlor, B., Segurado, R., Kennelly, S., Olde Rikkert, M. G. M., Howard, R., Pasquier, F., Borjesson-Hanson, A., Tsolaki, M., Lucca, U., Molloy, D. W., et al. (2018). Nilvadipine in mild to moderate Alzheimer disease: A randomised controlled trial. PLOS Med 15, e1002660.
Lee, C. Y. D., Daggett, A., Gu, X., Jiang, L. L., Langfelder, P., Li, X., Wang, N., Zhao, Y., Park, C. S., Cooper, Y., et al. (2018). Elevated TREM2 Gene Dosage Reprograms Microglia Responsivity and Ameliorates Pathological Phenotypes in Alzheimer's Disease Models. Neuron 97, 1032-1048 e1035.
Lionakis, M. S., Iliev, I. D., and Hohl, T. M. (2017). Immunity against fungi. JCI Insight 2.
Loving, B. A., Tang, M., Neal, M. C., Gorkhali, S., Murphy, R., Eckel, R. H., and Bruce, K. D. (2021). Lipoprotein Lipase Regulates Microglial Lipid Droplet Accumulation. Cells 10.
Lumb, S., Fleischer, S. J., Wiedemann, A., Daridon, C., Maloney, A., Shock, A., and Dorner, T. (2016). Engagement of CD22 on B cells with the monoclonal antibody epratuzumab stimulates the phosphorylation of upstream inhibitory signals of the B cell receptor. J Cell Commun Signal 10, 143-151.
Machhi, J., Yeapuri, P., Lu, Y., Foster, E., Chikhale, R., Herskovitz, J., Namminga, K. L., Olson, K. E., Abdelmoaty, M. M., Gao, J., et al. (2021). CD4+ effector T cells accelerate Alzheimer's disease in mice. J Neuroinflammation 18, 272.
Malik, A., Sharma, D., Malireddi, R. K. S., Guy, C. S., Chang, T. C., Olsen, S. R., Neale, G., Vogel, P., and Kanneganti, T. D. (2018). SYK-CARD9 Signaling Axis Promotes Gut Fungi-Mediated Inflammasome Activation to Restrict Colitis and Colon Cancer. Immunity 49, 515-530 e515.
Malik, M., Simpson, J. F., Parikh, I., Wilfred, B. R., Fardo, D. W., Nelson, P. T., and Estus, S. (2013). CD33 Alzheimer's risk-altering polymorphism, CD33 expression, and exon 2 splicing. J Neurosci 33, 13320-13325.
Marschallinger, J., Iram, T., Zardeneta, M., Lee, S. E., Lehallier, B., Haney, M. S., Pluvinage, J. V., Mathur, V., Hahn, O., Morgens, D. W., et al. (2020). Lipid-droplet-accumulating microglia represent a dysfunctional and proinflammatory state in the aging brain. Nat Neurosci 23, 194-208.
Martin, L. J., Pardo, C. A., Cork, L. C., and Price, D. L. (1994). Synaptic pathology and glial responses to neuronal injury precede the formation of senile plaques and amyloid deposits in the aging cerebral cortex. Am J Pathol 145, 1358-1381.
Mateo, I., Infante, J., Llorca, J., Rodriguez, E., Berciano, J., and Combarros, O. (2006). Association between glycogen synthase kinase-3beta genetic polymorphism and late-onset Alzheimer's disease. Dement Geriatr Cogn Disord 21, 228-232.
Mc Guire, C., Wieghofer, P., Elton, L., Muylaert, D., Prinz, M., Beyaert, R., and van Loo, G. (2013). Paracaspase MALTI deficiency protects mice from autoimmune-mediated demyelination. J Immunol 190, 2896-2903.
Mocsai, A., Ruland, J., and Tybulewicz, V. L. (2010). The SYK tyrosine kinase: a crucial player in diverse biological functions. Nat Rev Immunol 10, 387-402.
Molinero, L. L., Cubre, A., Mora-Solano, C., Wang, Y., and Alegre, M. L. (2012). T cell receptor/CARMA1/N F-kappaB signaling controls T-helper (Th) 17 differentiation. Proc Natl Acad Sci USA 109, 18529-18534.
Munawara, U., Catanzaro, M., Xu, W., Tan, C., Hirokawa, K., Bosco, N., Dumoulin, D., Khalil, A., Larbi, A., Levesque, S., et al. (2021). Hyperactivation of monocytes and macrophages in MCI patients contributes to the progression of Alzheimer's disease. Immun Ageing 18, 29.
Norris, G. T., Smirnov, I., Filiano, A. J., Shadowen, H. M., Cody, K. R., Thompson, J. A., Harris, T. H., Gaultier, A., Overall, C. C., and Kipnis, J. (2018). Neuronal integrity and complement control synaptic material clearance by microglia after CNS injury. J Exp Med 215, 1789-1801.
Nussbaum, R. L., and Ellis, C. E. (2003). Alzheimer's disease and Parkinson's disease. N Engl J Med 348, 1356-1364.
Parakalan, R., Jiang, B., Nimmi, B., Janani, M., Jayapal, M., Lu, J., Tay, S. S., Ling, E. A., and Dheen, S. T. (2012). Transcriptome analysis of amoeboid and ramified microglia isolated from the corpus callosum of rat brain. BMC Neurosci 13, 64.
Paris, D., Ait-Ghezala, G., Bachmeier, C., Laco, G., Beaulieu-Abdelahad, D., Lin, Y., Jin, C., Crawford, F., and Mullan, M. (2014). The spleen tyrosine kinase (Syk) regulates Alzheimer amyloid-beta production and Tau hyperphosphorylation. J Biol Chem 289, 33927-33944.
Plastini, M. J., Desu, H. L., and Brambilla, R. (2020). Dynamic Responses of Microglia in Animal Models of Multiple Sclerosis. Front Cell Neurosci 14, 269.
Pluvinage, J. V., Haney, M. S., Smith, B. A. H., Sun, J., Iram, T., Bonanno, L., Li, L., Lee, D. P., Morgens, D. W., Yang, A. C., et al. (2019). CD22 blockade restores homeostatic microglial phagocytosis in ageing brains. Nature 568, 187-192.
Podlesny-Drabiniok, A., Marcora, E., and Goate, A. M. (2020). Microglial Phagocytosis: A Disease-Associated Process Emerging from Alzheimer's Disease Genetics. Trends Neurosci 43, 965-979.
Ramagopalan, S. V., Dobson, R., Meier, U. C., and Giovannoni, G. (2010). Multiple sclerosis: risk factors, prodromes, and potential causal pathways. Lancet Neurol 9, 727-739.
Reddy, P. H. (2013). Amyloid beta-induced glycogen synthase kinase 3beta phosphorylated VDAC1 in Alzheimer's disease: implications for synaptic dysfunction and neuronal damage. Biochim Biophys Acta 1832, 1913-1921.
Reed-Geaghan, E. G., Croxford, A. L., Becher, B., and Landreth, G. E. (2020). Plaque-associated myeloid cells derive from resident microglia in an Alzheimer's disease model. J Exp Med 217.
Richard, B. C., Kurdakova, A., Baches, S., Bayer, T. A., Weggen, S., and Wirths, O. (2015). Gene Dosage Dependent Aggravation of the Neurological Phenotype in the 5XFAD Mouse Model of Alzheimer's Disease. J Alzheimers Dis 45, 1223-1236.
Schaffer, B. A., Bertram, L., Miller, B. L., Mullin, K., Weintraub, S., Johnson, N., Bigio, E. H., Mesulam, M., Wiedau-Pazos, M., Jackson, G. R., et al. (2008). Association of GSK3β with Alzheimer disease and frontotemporal dementia. Arch Neurol 65, 1368-1374.
Schweig, J. E., Yao, H., Beaulieu-Abdelahad, D., Ait-Ghezala, G., Mouzon, B., Crawford, F., Mullan, M., and Paris, D. (2017). Alzheimer's disease pathological lesions activate the spleen tyrosine kinase. Acta Neuropathol Commun 5, 69.
Serbulea, V., Upchurch, C. M., Ahern, K. W., Bories, G., Voigt, P., DeWeese, D. E., Meher, A. K., Harris, T. E., and Leitinger, N. (2018). Macrophages sensing oxidized DAMPs reprogram their metabolism to support redox homeostasis and inflammation through a TLR2-Syk-ceramide dependent mechanism. Mol Metab 7, 23-34.
Steen, E., Terry, B. M., Rivera, E. J., Cannon, J. L., Neely, T. R., Tavares, R., Xu, X. J., Wands, J. R., and de la Monte, S. M. (2005). Impaired insulin and insulin-like growth factor expression and signaling mechanisms in Alzheimer's disease—is this type 3 diabetes? J Alzheimers Dis 7, 63-80.
Stine, W. B., Jungbauer, L., Yu, C., and LaDu, M. J. (2011). Preparing synthetic Abeta in different aggregation states. Methods Mol Biol 670, 13-32.
Takahashi, K., Prinz, M., Stagi, M., Chechneva, O., and Neumann, H. (2007). TREM2-transduced myeloid precursors mediate nervous tissue debris clearance and facilitate recovery in an animal model of multiple sclerosis. PLOS Med 4, e124.
Taylor, J. P., Hardy, J., and Fischbeck, K. H. (2002). Toxic proteins in neurodegenerative disease. Science 296, 1991-1995.
Trapp, B. D., and Nave, K. A. (2008). Multiple sclerosis: an immune or neurodegenerative disorder? Annu Rev Neurosci 31, 247-269.
Ulland, T. K., Song, W. M., Huang, S. C., Ulrich, J. D., Sergushichev, A., Beatty, W. L., Loboda, A. A., Zhou, Y., Cairns, N. J., Kambal, A., et al. (2017). TREM2 Maintains Microglial Metabolic Fitness in Alzheimer's Disease. Cell 170, 649-663 e613.
Underhill, D. M., and Goodridge, H. S. (2007). The many faces of ITAMs. Trends Immunol 28, 66-73.
Van Hove, H., Martens, L., Scheyltjens, I., De Vlaminck, K., Pombo Antunes, A. R., De Prijck, S., Vandamme, N., De Schepper, S., Van Isterdael, G., Scott, C. L., et al. (2019). A single-cell atlas of mouse brain macrophages reveals unique transcriptional identities shaped by ontogeny and tissue environment. Nat Neurosci 22, 1021-1035.
Vickers, J. C., King, A. E., Woodhouse, A., Kirkcaldie, M. T., Staal, J. A., McCormack, G. H., Blizzard, C. A., Musgrove, R. E., Mitew, S., Liu, Y., et al. (2009). Axonopathy and cytoskeletal disruption in degenerative diseases of the central nervous system. Brain Res Bull 80, 217-223.
Walker, D. G., and Lue, L. F. (2015). Immune phenotypes of microglia in human neurodegenerative disease: challenges to detecting microglial polarization in human brains. Alzheimers Res Ther 7, 56.
Wang, Y., Cella, M., Mallinson, K., Ulrich, J. D., Young, K. L., Robinette, M. L., Gilfillan, S., Krishnan, G. M., Sudhakar, S., Zinselmeyer, B. H., et al. (2015). TREM2 lipid sensing sustains the microglial response in an Alzheimer's disease model. Cell 160, 1061-1071.
Wang, Y., Ulland, T. K., Ulrich, J. D., Song, W., Tzaferis, J. A., Hole, J. T., Yuan, P., Mahan, T. E., Shi, Y., Gilfillan, S., et al. (2016). TREM2-mediated early microglial response limits diffusion and toxicity of amyloid plaques. J Exp Med 213, 667-675.
Weinger, J. G., Brosnan, C. F., Loudig, O., Goldberg, M. F., Macian, F., Arnett, H. A., Prieto, A. L., Tsiperson, V., and Shafit-Zagardo, B. (2011). Loss of the receptor tyrosine kinase Axl leads to enhanced inflammation in the CNS and delayed removal of myelin debris during experimental autoimmune encephalomyelitis. J Neuroinflammation 8, 49.
Whittaker Hawkins, R. F., Patenaude, A., Dumas, A., Jain, R., Tesfagiorgis, Y., Kerfoot, S., Matsui, T., Gunzer, M., Poubelle, P. E., Larochelle, C., et al. (2017). ICAM1+neutrophils promote chronic inflammation via ASPRV1 in B cell-dependent autoimmune encephalomyelitis. JCI Insight 2.
Wissfeld, J., Nozaki, I., Mathews, M., Raschka, T., Ebeling, C., Hornung, V., Brustle, O., and Neumann, H. (2021). Deletion of Alzheimer's disease associated CD33 results in an inflammatory human microglia phenotype. Glia 69, 1393-1412.
Wu, X., Saito, T., Saido, T. C., Barron, A. M., and Ruedl, C. (2021). Microglia and CD206 (+) border-associated mouse macrophages maintain their embryonic origin during Alzheimer's disease. Elife 10.
Yao, H., Coppola, K., Schweig, J. E., Crawford, F., Mullan, M., and Paris, D. (2019). Distinct Signaling Pathways Regulate TREM2 Phagocytic and NFkappaB Antagonistic Activities. Front Cell Neurosci 13, 457.
Ye, X. C., Hao, Q., Ma, W. J., Zhao, Q. C., Wang, W. W., Yin, H. H., Zhang, T., Wang, M., Zan, K., Yang, X. X., et al. (2020). Dectin-1/Syk signaling triggers neuroinflammation after ischemic stroke in mice. J Neuroinflammation 17, 17.
Zhan, J., Mann, T., Joost, S., Behrangi, N., Frank, M., and Kipp, M. (2020). The Cuprizone Model: Dos and Do Nots. Cells 9.
Zhong, L., Xu, Y., Zhuo, R., Wang, T., Wang, K., Huang, R., Wang, D., Gao, Y., Zhu, Y., Sheng, X., et al. (2019). Soluble TREM2 ameliorates pathological phenotypes by modulating microglial functions in an Alzheimer's disease model. Nat Commun 10, 1365.
Ziegenfuss, J. S., Biswas, R., Avery, M. A., Hong, K., Sheehan, A. E., Yeung, Y. G., Stanley, E. R., and Freeman, M. R. (2008). Draper-dependent glial phagocytic activity is mediated by Src and Syk family kinase signalling. Nature 453, 935-939.
Zotova, E., Holmes, C., Johnston, D., Neal, J. W., Nicoll, J. A., and Boche, D. (2011). Microglial alterations in human Alzheimer's disease following Abeta42 immunization. Neuropathol Appl Neurobiol 37, 513-524.

### Example II

### Introduction

Tauopathies, which include frontotemporal dementia, Alzheimer's disease (AD), and chronic traumatic encephalopathy, are a class of neurological disorders resulting from pathogenic intracellular tau aggregates (1). Activation of microglia, the brain's residential immune cell, has broadly been shown to exacerbate pathology in many experimental models, as pro-inflammatory responses generated by aberrant innate immune activity trigger unchecked activation of tau kinases and a subsequent cascade involving tau hyperphosphorylation, tau tangle formation, and neuronal dysfunction (2) remain poorly defined, but a recent report from characterized the role of spleen tyrosine kinase (SYK) signaling in activating microglia in AD and EAE (3). It is hypothesized that SYK signaling will activate microglia and induce heighted neurotoxicity during tauopathies.

### Materials and Methods

Methods adapted from Ennerfelt et al., 2022 (3). Mice: Male Tau P301S (PS19+/−) mice were used in this study. To achieve genetic deletion of SYK in microglial, the Cx3cr1^{CreERT2} system was used. These mice were crossed generating PS19+/−; Syk^{fl/fl}, Cx3cr1^{CreERT2}_{-/-} (PS19 SYK^{WT}) and PS19+/−; Syk^{fl/fl}, Cx3cr1^{CreERT2}+/− (PS19 SYK^{ΔMG}). All mice were fed tamoxifen chow for 2 weeks at weaning and returned to regular chow for the remainder of the study. Morris Water Maze: Mice habituated to room for 1 hour. Training was 4 days long with 4 random drop locations/day with the platform submerged in opaque water. IHC and Microscopy: Free floating 50 μm sections were blocked with Mouse on Mouse (M.O.M.) Blocking Reagent. Primary antibodies were incubated overnight at 4° and secondaries at RT for 2 hours. All mounted sections were treated with True Black. ELISA: Tau (Phospho) (pT181) Human ELISA Kit was used according to manufactures instructions. Thawed soluble (PBS extracted) and insoluble (guanadine-HCl extracted) fractions were standardized to total protein loaded. Cortical samples were loaded at a concentration of 300 μg/ml and 3 μg/ml, respectively; hippocampal samples at 100 μg/ml and 1 μg/ml, respectively. Multiplex: Bio-Plex Multiplex System was used according to manufacturer's instructions. Statistical Tests: MWM was analyzed by a two-way repeated measures ANOVA on Prism. Imaging data was analyzed by R Studio using a linear mixed effects (LME) model to account for multiple images taking of each mouse. Data was explained by genotype and location with a random effect of mouse. Multiplex data utilized a Wilcoxon Rank Sum test with Holms' adjustment on R studio.

### Results

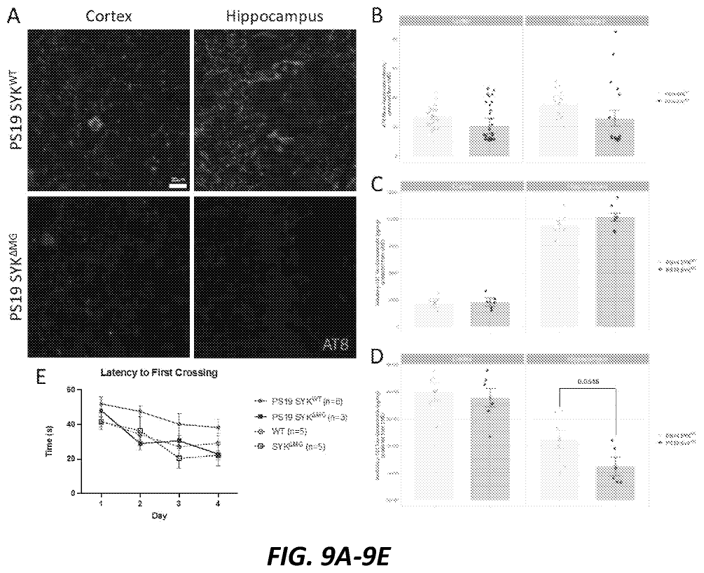

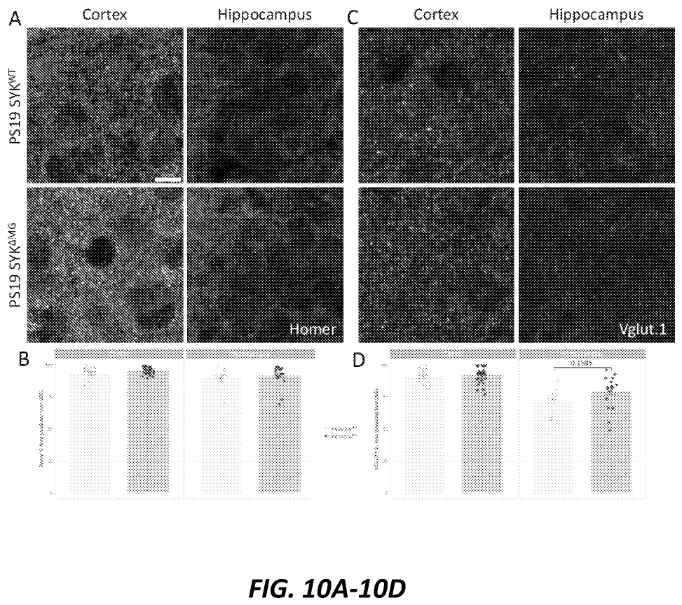

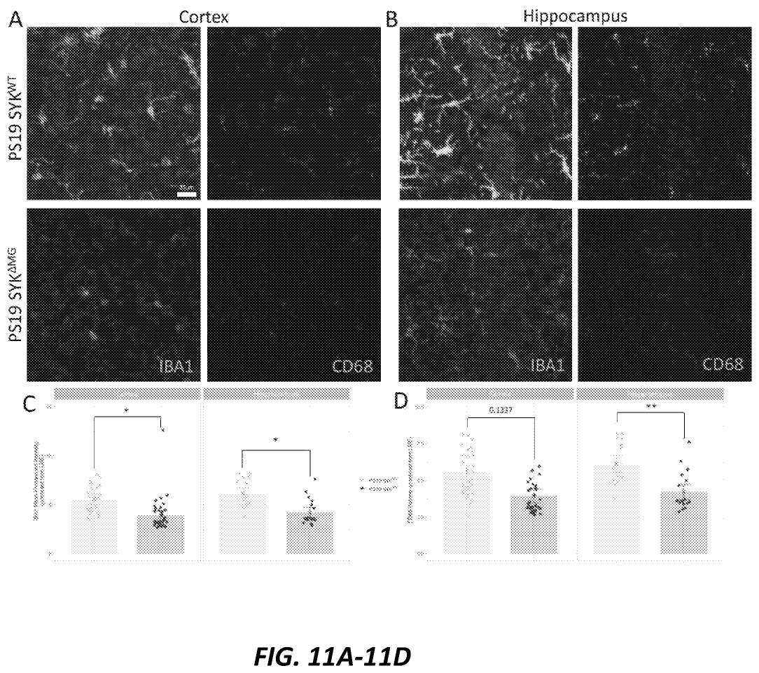

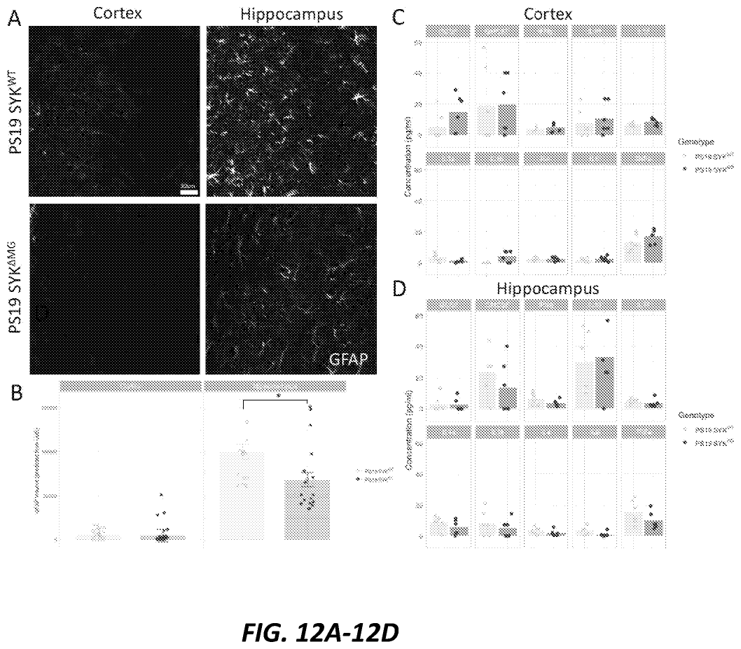

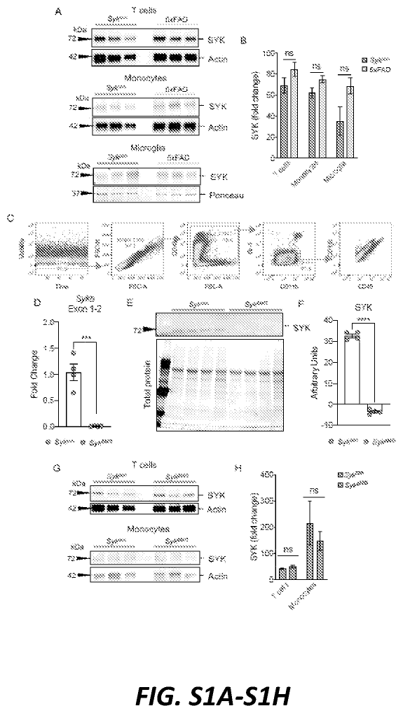

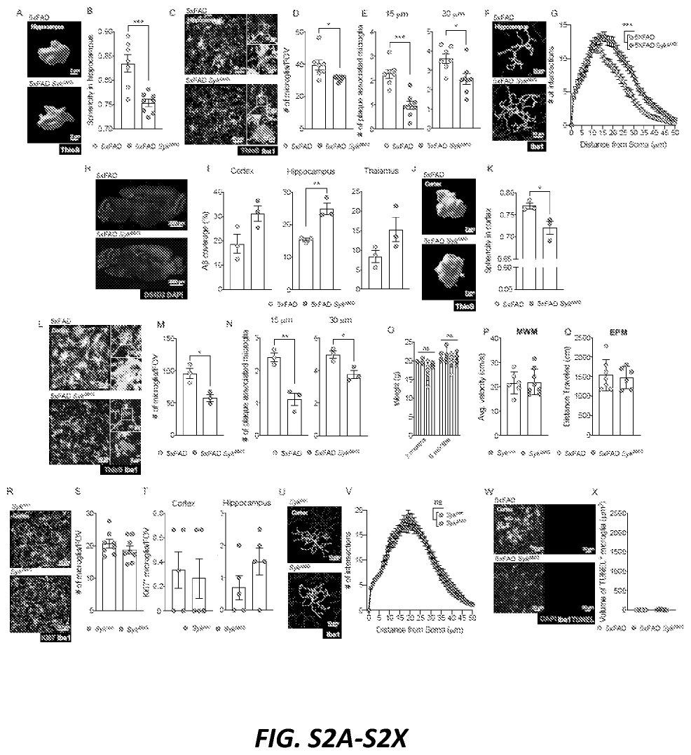

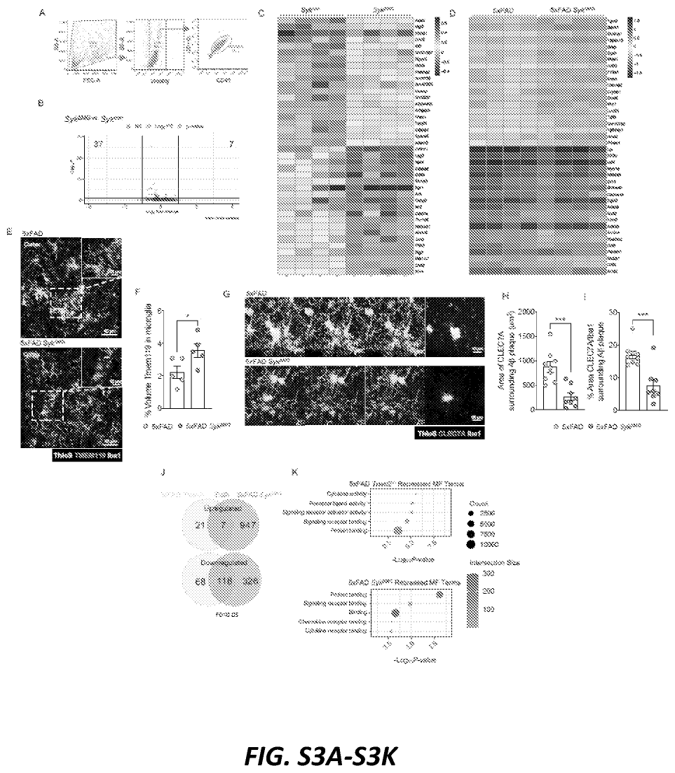

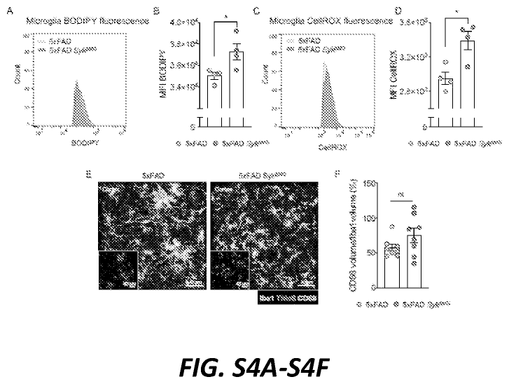

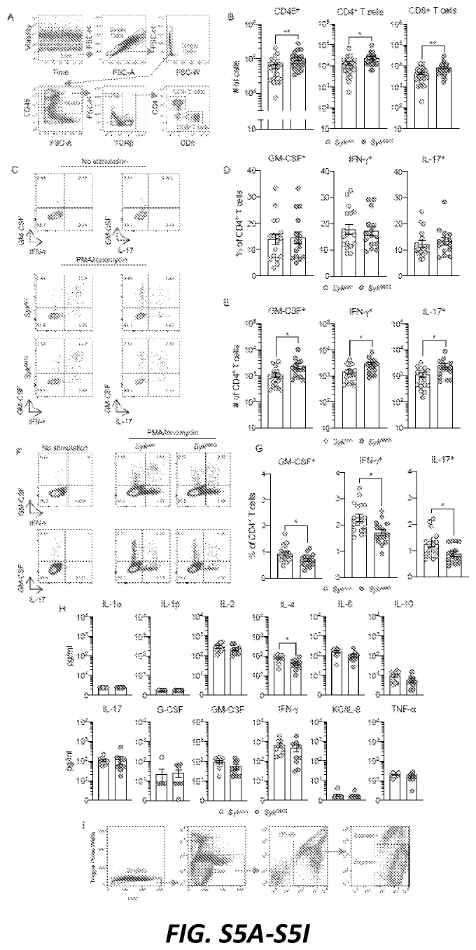

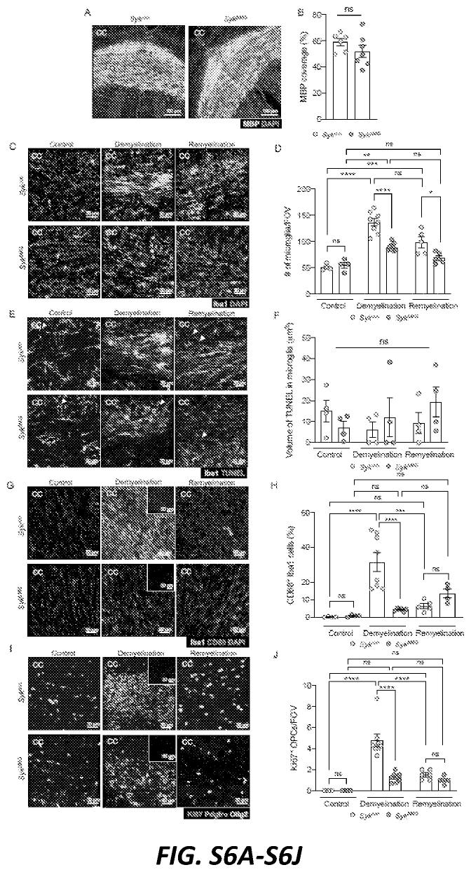

Microglial specific SYK deletion dampens hippocampal p-tau burden in 9 mo. male PS19 mice (FIG. 9). No significant differences observed in synaptic coverage of PS19 SYK^{ΔMG} mice relative to PS19 SYK^{WT} controls (FIG. 10). Microgliosis is significantly reduced in aged PS19 SYK^{ΔMG} mice (FIG. 11). Microglial SYK deletion has significant cell non-autonomous effects (FIG. 12).

### Discussion

Microglial SYK signaling propagates neurotoxic inflammation within the PS19 model of tauopathies. SYK signaling exacerbates increased hippocampal p-tau burden. Genetic ablation of microglial Syk rescues learning deficits. Loss of microglial SYK may not protect against synaptic loss. Gross microgliosis, measured by IBA1 fluorescence intensity and CD68 volume, is diminished in the absence of microglial SYK. The neuroinflammatory milieu is not significantly changed between PS19 SYK^{WT} and PS19 SYK^{ΔMG} mice. Hippocampal astrogliosis is drastically reduced in the absence of microglial SYK.

Thus, the data demonstrates that SYK inhibition is beneficial to treat tauopathy. This is opposite of what is seen in amyloid beta-mediated and demyelinating neurological diseases. Taken together, the work demonstrates that one would pharmacologically activate SYK in amyloid beta-mediated and demyelinating neurological diseases, whereas one would conversely inhibit SYK to treat tauopathies.

### BIBLIOGRAPHY FOR EXAMPLE II

1 Zhang, Y., Wu, K. M., Yang, L., Dong, Q., & Yu, J. T. (2022). Tauopathies: New perspectives and challenges. Molecular Neurodegeneration, 17(1), 28.
2 Johnson, A. M., & Lukens, J. R. (2023). The innate immune response in tauopathies. European Journal of Immunology, 2250266.
3 Ennerfelt, H., Frost, E. L., Shapiro, D. A., Holliday, C., Zengeler, K. E., Voithofer, G., . . . & Lukens, J. R. (2022). SYK coordinates neuroprotective microglial responses in neurodegenerative disease. Cell, 185(22), 4135-4152.
4 Bates, D., Mächler, M., Bolker, B., & Walker, S. (2015). Fitting Linear Mixed-Effects Models Using lme4. Journal of Statistical Software, 67(1), 1-48. https://doi.org/10.18637/jss.v067.i01

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

1. A method to treat or prevent Aβ-driven neurodegeneration or a demyelinating disease comprising administering an activator of SYK to a subject in need thereof.

2. (canceled)

3. (canceled)

4. (canceled)

5. (canceled)

6. A method to generate an increase in spatial learning and/or memory in Aβ-driven neurodegeneration or a demyelinating disease comprising administering an activator of SYK to a subject in need thereof.

7. (canceled)

8. The method of claim 1, wherein the subject is a mammal.

9. The method of claim 8, wherein the mammal is a human.

10. The method of claim 1, wherein the SYK activator comprises a β-D-glucan.

11. The method of claim 1, wherein the SYK activator comprises a ligand for CLEC7A.

12. The method of claim 1, wherein the SYK activator comprises pustulan.

13. The method of claim 1, wherein the Aβ-driven neurodegeneration is Alzheimer's Disease (AD).

14. The method of claim 1, wherein the demyelinating disease is multiple sclerosis (MS) Balo's disease, acute-disseminated encephalomyelitis, progressive multifocal leukoencephalopathy, Charcot-Marie-Tooth Disease, Guillain-Barre Syndrome (GBS), HTLV-I Associated Myelopathy (HAM), Neuromyelitis Optica (Devic's Disease), Schilder's Disease, Transverse Myelitis or extrapontine myelinolysis.

15. A method to treat or prevent a tauopathy comprising administering an inhibitor of SYK to a subject in need thereof.

16. The method of claim 15, wherein the subject is a mammal.

17. The method of claim 16, wherein the mammal is a human.

18. The method of claim 15, wherein the SYK inhibitor comprises GSK 143, Gusacitinib, R406, Fostamatinib, Piceatannol, Dehydroabietic acid, Cerdulatinib, MNS (3,4-Methylenedioxy-β-nitrostyrene), R112, BAY-61-3606, RO9021, Entospletinib, PRT-060318 2HCl, PRT062607 (P505-15) HCl, TAK-659, SRX3207, Lanraplenib or salts, compositions and combinations thereof.

19. The method of claim 15, wherein the tauopathy comprises Pick disease, progressive supranuclear palsy, corticobasal degeneration, argyrophilic grain disease, frontotemporal lobar degeneration (FTLD-Tau), Alzheimer disease (AD; a secondary tauopathy), chronic traumatic encephalopathy, post-encephalitic parkinsonism or Parkinsonism linked to chromosome 17.


SEQUENCE LISTING (1 sequence)

SEQ ID NO: 1 - protein, 635 aa, Homo sapiens
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN